(19) United States
(12) Patent Application Publication
(10) Pub. No.: US 2026/0364113 A1
(43) Pub. Date: Nov. 19, 2026

(54) CONDITIONING PROTOCOLS FOR USE WITH ANTI-VIRAL CENTRAL MEMORY CD8+ VETO CELLS IN HAPLOIDENTICAL STEM CELL TRANSPLANTATION

(71) Applicants: Yeda Research and Development Co. Ltd., Rehovot (IL); Board of Regents, The University of Texas System, Austin, TX (US)

(72) Inventors: Yair REISNER, Houston, TX (US); Esther BACHAR-LUSTIG, Rehovot (IL)

(73) Assignees: Yeda Research and Development Co. Ltd., Rehovot (IL); Board of Regents, The University of Texas System, Austin, TX (US)

(21) Appl. No.: 19/125,746

(22) PCT Filed: Oct. 31, 2023
(86) PCT No.: PCT/IL2023/051121

§ 371 Date: Apr. 30, 2025

Related U.S. Application Data
(60) Provisional application No. 63/420,741, filed on Oct. 31, 2022.

(51) Int. Cl. A61K 35/28; A61K 31/675; A61K 31/7076; A61N 5/10; A61P 35/02; A61P 37/06; C12N 5/00; C12N 5/0789

(52) U.S. Cl. CPC A61K 35/28; A61K 31/675; A61K 31/7076; A61N 5/10; A61P 35/02; A61P 37/06; C12N 5/0018; C12N 5/0087; C12N 5/0647; C12N 2501/2307; C12N 2501/2315; C12N 2501/2321

(57) ABSTRACT

Conditioning protocols for use with anti-viral central memory CD8+ veto cells in haploidentical stem cell transplantation are provided. Accordingly, there is provided a method of treating a disease in a subject in need thereof, wherein the disease comprises pathological cells residing in a bone marrow of the subject, the method comprising: conditioning the subject under a pre-transplant conditioning protocol comprising total marrow irradiation; transplanting into the subject T cell depleted immature hematopoietic cells; administering to the subject cyclophosphamide; and administering to the subject veto non-graft versus host disease inducing cells.

### RELATED APPLICATION/S

This application claims the benefit of priority of U.S. Provisional Patent Application No. 63/420,741 filed on Oct. 31, 2022, the contents of which are incorporated herein by reference in their entirety.

### FIELD AND BACKGROUND OF THE INVENTION

The present invention, in some embodiments thereof, relates to conditioning protocols for use with anti-viral central memory CD8+ veto cells in haploidentical stem cell transplantation.

The paradigm that high dose chemoradiotherapy “conditioning” is critical for attaining durable remission with allogeneic hematopoietic cell transplantation (allo-HCT) for hematological malignancies has shifted to emphasize the immune graft-vs-malignancy effect of the newly formed, donor derived immune system, in conjunction with subsequent immunotherapy. However, reduction of the conditioning, which retains a more robust host immune system, capable of protecting the patient from lethal infection during the early period post-transplant, is also associated with enhanced risk for graft rejection. In Haploidentical HCT, this can be overcome by using T cell replete transplants, as the alloreactive T cells in the graft are capable of attacking host T cells and paving the way for engraftment of the donor stem cells. However, such alloreactive T cells are also associated with life-threatening graft-versus-host (GVH) reactivity, requiring use of potent immunosuppressive drugs post-transplant. Some progress has been made in recent years in reducing the severity of graft-versus-host disease (GVHD) using high dose cyclophosphamide (CY) following transplant. Nevertheless, chronic GVHD remains a problem and prolonged use of immuno-suppressive drugs is required, which adversely impacts graft-vs-malignancy effects. Furthermore, the occurrence of GVHD in many patients is associated with poor thymic function, further limiting adequate anti-tumor immunity.

To overcome this obstacle, ‘megadose’ T cell depleted haploidentical HCT may be used, which is free of GVHD risk even in the absence of post-transplant immune suppressive therapy. However, achieving engraftment after non-myeloablative (NMA) conditioning remains a major challenge due to the high level of anti-donor T cell clones surviving the pre-transplant conditioning regimen. One approach used to address this challenge is the use of high dose CY shortly following transplant (discussed in PCT publication nos. WO 2013/093920 and WO 2013/093919).

In addition, various approaches have been contemplated for generation of tolerance inducing cells (e.g. veto cells) devoid of GVH reactivity and the use of same as an adjuvant treatment for graft transplantation (see e.g. PCT Publication Nos. WO 2001/49243, WO 2007/023491, WO 2010/049935, WO 2012/032526, WO 2013/035099, WO 2018/002924, WO 2017/009852, WO 2017/009853, WO2018/134824). Furthermore, a strategy combining non-myeloablative conditioning with T cell-depleted megadose HCT and veto cells has been described (PCT Publication No. WO 2021/024264).

Total marrow irradiation (TMI) has been introduced as part of conditioning regimens prior to hematopoietic cell transplantation with the aim of reducing toxicities induced by total body irradiation (TBI). TMI focuses the dose to the entire skeleton, while sparing the rest of the body, allowing dose escalation to the bone marrow with acceptable toxicity. TMI has been suggested for example as part of reduced-intensity systemic conditioning regimens, where malignant cytoreduction is still necessary; incorporated into conditioning regimens as a strategy to provide a myeloablative dose of radiation to the total marrow; and added to regimens that use strategies to reduce transplant related mortality [e.g. Jensen et al. (Biol Blood Marrow Transplant, 2018, 24(2):301-307) and Welliver et al. (International Journal of Radiation Oncology, Biology, Physics, 2018, 102(3): e370-e371).

### SUMMARY OF THE INVENTION

According to an aspect of some embodiments of the present invention there is provided a method of treating a disease in a subject in need thereof, wherein the disease comprises pathological cells residing in a bone marrow of the subject, the method comprising:
(a) conditioning the subject under a pre-transplant conditioning protocol comprising a therapeutically effective amount of total marrow irradiation (TMI); and subsequently
(b) transplanting into the subject therapeutically effective amount of T cell depleted immature hematopoietic cells; and subsequently
(c) administering to the subject a therapeutically effective amount of cyclophosphamide; and subsequently
(d) administering to the subject a therapeutically effective amount of veto non-graft versus host disease (GVHD) inducing cells, thereby treating the disease in the subject.

According to an aspect of some embodiments of the present invention there is provided a combination of pre-transplant conditioning protocol comprising a therapeutically effective amount of total marrow irradiation (TMI), a therapeutically effective amount of T cell depleted immature hematopoietic cells, a therapeutically effective amount of cyclophosphamide and a therapeutically effective amount of veto non-graft versus host disease (GVHD) inducing cells for use in treating a disease in a subject in need thereof, wherein the disease comprises pathological cells residing in a bone marrow of the subject.

According to some embodiments of the invention, the disease is cancer.

According to some embodiments of the invention, the cancer comprises a myeloid malignancy or multiple myeloma.

According to some embodiments of the invention, the myeloid malignancy is selected from the group consisting of AML, CML and MDS.

According to some embodiments of the invention, the cancer comprises AML or MDS.

According to some embodiments of the invention, the disease is non-cancerous.

According to some embodiments of the invention, the disease is selected from the group consisting of sickle cell anemia, aplastic anemia, thalassemia and metabolic genetic disease.

According to some embodiments of the invention, the veto non-GVHD inducing cells are obtained from the same donor as the T cell depleted immature hematopoietic cells.

According to some embodiments of the invention, the conditioning further comprises a therapeutically effective amount of spleen irradiation.

According to some embodiments of the invention, the TMI is administered on days −7 to −1 prior to transplantation of the T cell depleted immature hematopoietic cells.

According to some embodiments of the invention, the therapeutically effective amount of the TMI comprises a total of 8-18 Gy.

According to some embodiments of the invention, the therapeutically effective amount of the TMI is administered in at least 2 doses administered on consecutive days.

According to some embodiments of the invention, the therapeutically effective amount of the TMI is administered in 4 doses administered on consecutive days.

According to some embodiments of the invention, the T cell depleted immature hematopoietic cells are derived from a donor non-syngeneic to the subject.

According to some embodiments of the invention, the non-syngeneic is allogeneic.

According to some embodiments of the invention, the allogeneic donor is an HLA matched sibling, an HLA matched unrelated donor, an HLA haploidentical related donor or a donor displaying one or more disparate HLA determinants.

According to some embodiments of the invention, the therapeutically effective amount of the T cell depleted immature hematopoietic cells comprises less than 5×10⁵ CD3⁺ T cells per kilogram ideal body weight of the subject.

According to some embodiments of the invention, the therapeutically effective amount of the T cell depleted immature hematopoietic cells comprises less than 2×10⁵ CD3⁺ T cells per kilogram ideal body weight of the subject.

According to some embodiments of the invention, the therapeutically effective amount of the T cell depleted immature hematopoietic cells comprises at least 5×10⁶ CD34⁺ cells per kilogram ideal body weight of the subject.

According to some embodiments of the invention, the therapeutically effective amount of the T cell depleted immature hematopoietic cells are depleted of CD3⁺ and/or CD19⁺ expressing cells.

According to some embodiments of the invention, the therapeutically effective amount of the cyclophosphamide comprises 25-200 mg cyclophosphamide per kilogram ideal body weight of the subject.

According to some embodiments of the invention, the therapeutically effective amount of the cyclophosphamide is administered to the subject in two doses between days 2 and 5 following the transplantation of the T cell depleted immature hematopoietic cells.

According to some embodiments of the invention, the therapeutically effective amount of the cyclophosphamide is administered to the subject in two doses 3 and 4 days following the transplantation of the T cell depleted immature hematopoietic cells.

According to some embodiments of the invention, the veto non-GVHD inducing cells comprise a central memory T-lymphocyte (Tcm) phenotype.

According to some embodiments of the invention, the veto non-GVHD inducing cells have an anti-viral activity.

According to some embodiments of the invention, the veto non-GVHD inducing cells are obtainable by:
(i) contacting a first population of peripheral blood mononuclear cells (PBMCs) from a donor with an antibody capable of binding CD14⁺ expressing cells and selecting CD14⁺ expressing cells capable of maturing into antigen presenting cells;
(ii) loading the antigen presenting cells with a viral peptide;
(iii) treating a second population of PBMCs of the same donor as the first population of PBMCs with one or more agents capable of depleting CD4⁺, CD56⁺ and CD45RA⁺ expressing cells so as to obtain a population of cells comprising T cells enriched in memory T cells expressing a CD45RA⁻CD8⁺ phenotype;
(iv) contacting the population of cells comprising the T cells enriched in the memory T cells with the antigen presenting cells loaded with the viral peptides of step (ii) in the presence of IL-21 so as to allow enrichment of viral reactive memory T cells; and
(v) culturing the cells resulting from step (iv) in the presence of IL-21, IL-15 and/or IL-7 so as to allow proliferation of cells comprising the Tcm phenotype.

According to some embodiments of the invention, the therapeutically effective amount of the veto non-GVHD inducing cells is administered on day 6-9 following the transplantation of the T cell depleted immature hematopoietic cells.

According to some embodiments of the invention, the therapeutically effective amount of the veto non-GVHD inducing cells comprises at least 2.5×10⁶ CD8⁺ cells per kg ideal body weight of the subject.

According to some embodiments of the invention, the subject is not treated chronically with GVHD prophylaxis following the transplantation.

According to some embodiments of the invention, the conditioning further comprises an anti-B cell therapy.

According to some embodiments of the invention, the anti-B cell therapy comprises an anti-B cell antibody.

According to some embodiments of the invention, the anti-B cell therapy comprises Rituximab.

According to some embodiments of the invention, the conditioning further comprises T cell debulking.

According to some embodiments of the invention, the T cell debulking is effected by antibodies, and optionally wherein the antibodies comprise at least one of an anti-thymocyte globulin (ATG) antibody, an anti-CD52 antibody and anti-CD3 antibody.

According to some embodiments of the invention, the T cell debulking is effected by an anti-thymocyte globulin (ATG) antibody.

According to some embodiments of the invention, the conditioning further comprises a chemotherapeutic agent.

According to some embodiments of the invention, the chemotherapeutic agent comprises at least one of Fludarabine, Busulfan, Melphalan, Thiotepa and cyclophosphamide.

According to some embodiments of the invention, the chemotherapeutic agent comprises Fludarabine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of the treatment protocol according to some embodiments of the invention. Optionally, the protocol also includes 375 mg/m² Rituximab on day −15.

FIG. 2 is a flow diagram outlining processing and testing of anti-viral CD8⁺ veto cell depleted cells according to some embodiments of the invention.

### DESCRIPTION OF SPECIFIC EMBODIMENTS OF THE INVENTION

The present invention, in some embodiments thereof, relates to conditioning protocols for use with anti-viral central memory CD8+ veto cells in haploidentical stem cell transplantation.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Myelodysplastic syndrome (MDS) and acute myeloid leukemia (AML) are cancerous diseases leading to an insufficient hematopoiesis. Chronic fatigue due to anemia, bleeding due to thrombocytopenia and infection due to neutropenia are typical consequences of these diseases (Weber et al. Front Immunol. 2020; 11:627662). Despite increasing knowledge and development of novel drugs and treatment protocols the overall survival rate of subjects diagnosed with these diseases is still low. For example, the 5-year overall survival rate of AML patients is below 30% (Weber et al. Front Immunol. 2020; 11:627662).

While reducing to practice specific embodiments of the present invention, the present inventors developed a protocol for the treatment of cancer such as AML and MDS. Embodiments of the protocol comprise treating the subject with total marrow irradiation (TMI) and cyclophosphamide coupled with transplanting the subject with a megadose T cell depleted allogeneic HSC transplant and anti-viral central memory CD8⁺ veto cells (see Examples 1-3 of the Examples section which follows). This protocol is expected to be a game changer in allogeneic HSC transplantations in the treatment of diseases such as cancer that comprise pathological cells residing in the bone marrow of the subject.

Thus, according to an aspect of the present invention there is provided a method of treating a disease in a subject in need thereof, wherein said disease comprises pathological cells residing in a bone marrow of said subject, the method comprising:
(a) conditioning the subject under a pre-transplant conditioning protocol comprising a therapeutically effective amount of total marrow irradiation (TMI); and subsequently
(b) transplanting into said subject therapeutically effective amount of T cell depleted immature hematopoietic cells; and subsequently
(c) administering to said subject a therapeutically effective amount of cyclophosphamide; and subsequently
(d) administering to said subject a therapeutically effective amount of veto non-graft versus host disease (GVHD) inducing cells,
thereby treating the disease in the subject.

According to an additional or an alternative aspect of the present invention there is provided a combination of pre-transplant conditioning protocol comprising a therapeutically effective amount of total marrow irradiation (TMI), a therapeutically effective amount of T cell depleted immature hematopoietic cells, a therapeutically effective amount of cyclophosphamide and a therapeutically effective amount of veto non-graft versus host disease (GVHD) inducing cells for use in treating a disease in a subject in need thereof, wherein said cancer comprises pathological cells residing in a bone marrow of said subject.

As used herein, the term “treating” includes abrogating, substantially inhibiting, slowing or reversing the progression of a condition (i.e. disease e.g. cancer), substantially ameliorating clinical or aesthetical symptoms of a condition or substantially preventing the appearance of clinical or aesthetical symptoms of a condition.

According to specific embodiments, the treatment protocol described herein is the first line of treatment.

According to specific embodiments, the treatment protocol described herein is at least the second line of treatment.

According to specific embodiments, the treatment protocol described herein is at least the third line of treatment.

As used herein, the term “subject” or “subject in need thereof” refers to a mammal, preferably a human being, of any gender and at any age that is diagnosed with the disease as defined herein.

According to specific embodiments, the subject is a human subject.

According to specific embodiments, the subject is at least 12, at least 15, at least 20, at least 30, at least 40, at least 50, at least 60, or at least 70 years old, each possibility represents a separate embodiment of the invention.

According to specific embodiments, the subject is at least 12 years old.

According to specific embodiments, the subject is not a suitable candidate for pre-transplant conditioning protocol with direct total body irradiation (TBI, in which the entire body is directly exposed to the ionizing radiation) as it may not tolerate the procedure due to its associated toxic side effects.

According to specific embodiments, the subject is not a suitable candidate for pre-transplant conditioning protocol with direct TBI, as this procedure is considered overly aggressive for the treated disease.

According to specific embodiments, the subject has been treated with at least one cycle of treatment (i.e. distinct from the treatment protocol described herein) for the disease prior to treatment according to the method and uses described herein.

The methods and uses described herein are applied for the treatment of diseases comprising pathological cells residing in the bone marrow.

Methods of determining presence of such cell in the bone marrow are known in the art and include, but not limited to histology, immunocytochemistry, flow cytometry, western blot, single cell genomic analysis, PCR and the like.

According to specific embodiments, the pathological cells comprise less than 5%, less than 2%, less the 1%, less than 0.5%, less than 0.1%, less than 0.05% or less than 0.01% of BM cells.

According to specific embodiments, presence of the pathological cells in the BM is detected by histology, immunocytochemistry, flow cytometry, western blot, single cell genomic analysis and/or PCR determined on a BM aspiration or biopsy sample obtained from the subject.

According to specific embodiments, the disease is cancer.

Non-limiting examples of such cancers include myeloid malignancy [e.g. acute myeloid leukemia (AML), chronic myeloid leukemia (CML), myelodysplastic syndrome (MDS)] and multiple myeloma.

According to specific embodiments, the cancer comprises AML or MDS.

According to specific embodiments, the cancer is a primary cancer.

According to specific embodiments, the cancer is at least a secondary cancer.

According to specific embodiments, the cancer is a refractory or relapsed cancer.

According to specific embodiments, the disease is non-cancerous.

Non-limiting examples of such non-cancerous diseases include sickle cell anemia, aplastic anemia, thalassemia and metabolic genetic diseases.

Non-limiting examples of such metabolic genetic diseases are described for example in Tan et al. Front Pediatr. 2019; 7:433, the contents of which are fully incorporated herein by reference, and include e.g. Hurler, Hunter, Maroteaux-Lamy, Sly, MLD, GLD, Niemann pick, Tay Sachs, Sandhoff, Farber, Alpha-mannosidosis, Fucosidosis, Aspartylglycosminuria, Wolmann syndrome, Pompe.

The methods and uses described herein comprise a pre-transplant conditioning protocol comprising a therapeutically effective amount of total marrow irradiation (TMI).

The conditioning may be effected under sublethal, lethal or supralethal conditions prior to transplantation of the T cell depleted immature hematopoietic cells.

As used herein, the terms “sublethal”, “lethal”, and “supralethal”, when relating to conditioning of subjects, refer to myelotoxic and/or lymphocytotoxic treatments which, when applied to a representative population of the subjects, respectively, are typically: non-lethal to essentially all members of the population; lethal to some but not all members of the population; or lethal to essentially all members of the population under normal conditions of sterility.

According to specific embodiments, the conditioning is myeloablative.

According to specific embodiments, the conditioning is non-myeloablative.

“Total marrow irradiation (TMI)” refers to irradiation of the entire skeleton allowing penetration of radiation to the total marrow, while significantly minimizing exposure to the rest of the body. It is important to note that TMI may involve irradiation leakage into other body regions, accounting for about 25% of the total irradiation dose. However, according to embodiments of the invention, there is no active TBI application in the envisaged regimen. TMI was made possible through recent developments of modulated radiotherapy having the ability to provide radiation doses to large target regions while simultaneously reducing doses to non-target organs and organs at risk. Such methods include, but not limited to helical tomotherapy (HT), Intensity-Modulated Radiation Therapy (IMRT), volumetric arc based intensity modulated radiotherapy (VMAT), and 3D-conformal radiation therapy (3D-CRT).

Methods of effecting TMI are known in the art and disclosed e.g. in Jensen et al. (Biol Blood Marrow Transplant, 2018, 24(2):301-307) Welliver et al. (International Journal of Radiation Oncology, Biology, Physics, 2018, 102(3): e370-e371), Mancosu et al. (Physics and Imaging in Radiation Oncology 11 (2019) 47-53), Wong et al. (Lancet Oncol 2020; 21: e477-87), Chilukuril et al. (Radiat Oncol J 2020; 38(3):207-216), Haraldsson et al. Physica Medica 60 (2019) 162-167, the contents of which are all incorporated herein by reference, and are further described herein and in the Examples section which follows.

According to specific embodiments, TMI is effected with the aid of computed tomography (CT) scans to define the target organs, contour the organs at risk and calculate the doses.

Avoidance organs (i.e., off target organs) include for example lungs, heart, kidneys, liver, esophagus, oral cavity, parotid glands, thyroid gland, eyes, lens, brain, stomach, small bowel, rectum, bladder, testes etc.

Typically, when effecting TMI the subject is immobilized in a supine position.

According to specific embodiments, the subject is positioned in a whole body vacuum bag or cushion, the arms are relaxed, down by the sides, in a non-akimbo fashion, with fingers holding onto the bag. Additional devices may be used as needed for patient comfort to ensure positioning reproducibility such as face mask, neck support, cotton pads.

According to specific embodiments, the size of the dose grid should be ≤3 mm in all directions.

According to specific embodiments, TMI is effected with volumetric modulated arc therapy (VMAT) and/or 3D-conformal radiation therapy (3D-CRT).

According to a specific embodiments, TMI is effected with volumetric modulated arc therapy (VMAT) for the body and 3D-conformal radiation therapy (3D-CRT) for the legs.

As a non-limiting example, for the VMAT body plan, 6 to 12 overlapping arcs from 3 to 6 different isocenters may be used. Depending on patient height and anatomy, more arcs and isocenters may be needed for larger patients. The isocenters for these arcs and field sizes are determined based on patient anatomy to optimize the travel of the multileaf collimators. According to specific embodiments, for each arc, there is ≥2 cm overlap region with the arc superior to it, and another ≥2 cm overlap region with the arc inferior to it.

As a non-limiting example, for the 3D legs plan, the multileaf collimators conform to the PTV from the beam's-eye-views.

According to specific embodiments, there is ≥2 cm overlap region between the VMAT body plan and the 3D leg plan.

A non-limiting example of a radiation machine comprises a 6 megavoltage (MV) photons delivered from a TrueBeam (Varian Medical Systems, Palo Alto, California, U.S.) linear accelerator equipped with the Millennium 120 multileaf collimator.

A detailed description of an irradiation protocol that can be used with specific embodiments of the invention is provided in Example 1 of the Examples section which follows that serve as an integral part of the specification.

According to specific embodiments, the therapeutically effective amount of TMI comprises a total dose of 5-25 Gy, 5-20 Gy, 5-18 Gy, 8-18 Gy, 8-15 Gy, 10-15 Gy, or 11-14 Gy.

According to specific embodiments, the therapeutically effective amount of TMI comprises a total dose of 8-18 Gy.

According to a specific embodiment, the therapeutically effective amount of TMI comprises a total dose of about 12 Gy.

The conditioning may be effect by a single irradiation dose or fractionated irradiation doses.

Thus, according to specific embodiments, the subject is subjected to TMI at least 2, at least 3, at least 4 times.

According to one embodiment, the subject is subjected to TMI 4 times.

According to specific embodiments, the doses are administered on consecutive days.

According to a specific embodiment, the therapeutically effective amount of TMI is administered in at least 2 doses administered on consecutive days.

According to a specific embodiment, the therapeutically effective amount of TMI is administered in 4 doses administered on consecutive days.

According to specific embodiments, the first dose of TMI is administered to the subject at least 4, at least 5, at least 6, at least 7, or at least 10 days prior to transplantation of the T cell depleted hematopoietic cells.

According to specific embodiments, the last dose of TMI is administered to the subject no later than 1 day, no later than 2 days, no later than 3 days, no later than 4 days prior to transplantation of the T cell depleted hematopoietic cells.

According to specific embodiments, the TMI is administered to the subject on days −14 to −1, on days −14 to −3, on days −14 to −4, on days −10 to −1, on days −10 to −3, on days −10 to −4, on days-7 to −1, on days −7 to −3, or on days −7 to −4 prior to transplantation of the T cell depleted hematopoietic cells.

According to specific embodiments, TMI is administered on days −7 to −1 prior to transplantation of the T cell depleted immature hematopoietic cells.

According to one embodiment, the subject is subjected to TMI 4 times, on days −7, −6, −5 and −4 prior to transplantation of the T cell depleted hematopoietic cells.

According to one embodiment, the subject is subjected to TMI 4 times, on days −7, −6, −5 and −4 prior to transplantation of the T cell depleted hematopoietic cells, each in a dose of about 3Gy.

According to specific embodiments, conditioning further comprises a therapeutically effective amount of spleen irradiation.

The spleen irradiation may be effected in the same manner as TMI, using the same methodologies and machines marking the spleen as a target organ (as further described in details hereinabove and in the Examples section which follows).

According to specific embodiments, the therapeutically effective amount of spleen irradiation comprises a total dose of 5-25 Gy, 5-20 Gy, 5-18 Gy, 8-18 Gy, 8-15 Gy, 10-15 Gy, or 11-14 Gy.

According to specific embodiments, the therapeutically effective amount of spleen irradiation comprises a total dose of 8-18 Gy.

According to a specific embodiment, the therapeutically effective amount of spleen irradiation comprises a total dose of about 12 Gy.

The conditioning may be effect by a single spleen irradiation dose or fractionated irradiation doses.

According to specific embodiments, the spleen irradiation is effected concomitantly with TMI.

Thus, according to specific embodiments, the subject is subjected to spleen irradiation at least 2, at least 3, at least 4 times.

According to one embodiment, the subject is subjected to spleen irradiation 4 times.

According to specific embodiments, the doses are administered on consecutive days.

According to a specific embodiment, the therapeutically effective amount of spleen irradiation is administered in at least 2 doses administered on consecutive days.

According to a specific embodiment, the therapeutically effective amount of spleen irradiation is administered in 4 doses administered on consecutive days.

According to specific embodiments, the first dose of spleen irradiation is administered to the subject at least 4, at least 5, at least 6, at least 7, or at least 10 days prior to transplantation of the T cell depleted hematopoietic cells.

According to specific embodiments, the last dose of spleen irradiation is administered to the subject no later than 1 day, no later than 2 days, no later than 3 days, no later than 4 days prior to transplantation of the T cell depleted hematopoietic cells.

According to specific embodiments, the spleen irradiation is administered to the subject on days −14 to −1, on days −14 to −3, on days −14 to −4, on days −10 to −1, on days −10 to −3, on days −10 to −4, on days −7 to −1, on days −7 to −3, or on days −7 to −4 prior to transplantation of the T cell depleted hematopoietic cells.

According to specific embodiments, spleen irradiation is administered on days −7 to −1 prior to transplantation of the T cell depleted immature hematopoietic cells.

According to one embodiment, the subject is subjected to spleen irradiation 4 times, on days −7, −6, −5 and −4 prior to transplantation of the T cell depleted hematopoietic cells.

According to one embodiment, the subject is subjected to spleen irradiation 4 times, on days −7, −6, −5 and −4 prior to transplantation of the T cell depleted hematopoietic cells, each in a dose of about 3Gy.

According to specific embodiments, the conditioning further comprises a chemotherapeutic agent.

Exemplary chemotherapeutic agents include, but are not limited to, Busulfan, Busulfex, Cyclophosphamide, Fludarabine, Melphalan, Myleran, Rapamycin, Trisulphan, and Thiotepa.

According to specific embodiments, the chemotherapeutic agent comprises at least one of Fludarabine, Busulfan, Melphalan, Thiotepa and cyclophosphamide.

The chemotherapeutic agent/s may be administered to the subject in a single dose or in several doses e.g. 2, 3, 4, 5, 6, 7, 8, 9, 10 or more doses (e.g. daily doses) prior to transplantation.

According to a specific embodiment, the subject is administered a chemotherapeutic agent (e.g. Fludarabine e.g. at a dose of 20-40 mg/m²/day e.g. about 30 mg/m²/day) for 3, 4, 5 or 6 consecutive days (e.g. 4 consecutive days) prior to transplantation (e.g. on days −11 to −8 prior to transplantation of the T cell depleted immature hematopoietic cells).

Fludarabine is commercially available from e.g. Sanofi Genzyme, Bayer and Teva, e.g. under the brand name e.g. Fludara.

According to specific embodiments, the conditioning further comprises in vivo T cell debulking.

According to some embodiments, the in-vivo T cell debulking is affected by antibodies.

According to some embodiments of the invention, the antibodies comprise an anti-CD8 antibody, an anti-CD4 antibody, or both.

According to some embodiments of the invention, the antibodies comprise at least one of an anti-thymocyte globulin (ATG) antibody, an anti-CD52 antibody and anti-CD3 (e.g. OKT3) antibody.

Such antibodies are commercially available, e.g. ATG is commercially available from e.g. Genzyme and Pfizer, e.g. under the brand names e.g. Thymoglobulin and Atgam.

According to one embodiment, the antibody is administered to the subject in a single dose or in several doses e.g. 2, 3, 4, 5, 6, 7, 8, 9, 10 or more doses (e.g. daily doses) prior to transplantation.

According to a specific embodiment, the subject is administered an antibody therapeutic agent (e.g. ATG e.g. at a dose of 0.5-4 mg per kg ideal body weight e.g. about 2 mg per kg ideal body weight) for 2, 3, 4 or 5 consecutive days (e.g. 3 consecutive days) prior to transplantation (e.g. on days −18 to −12, on days −14 to −12, or on day −12 prior to transplantation of the T cell depleted immature hematopoietic cells).

According to a specific embodiment, the pre-transplant conditioning does not comprise in vivo T cell debulking.

According to a specific embodiment, the subject is not treated with ATG prior to transplantation.

It will be appreciated that when using no ATG or lower doses of ATG are used, e.g. single dose or two doses (e.g. each at a dose of about 2 mg per kg ideal body weight), higher radiation doses can be used as part of the conditioning protocol.

The term “ideal body weight”, as used herein, refers to the measurement used clinically to adjust drug dosing, help estimate renal function and the pharmacokinetics (such as in obese patients).

The formula for estimating ideal body weight in (kg) is as follows:
Males: IBW=50 kg+2.3 kg for each inch over 5 feet.
Females: IBW=45.5 kg+2.3 kg for each inch over 5 feet.

Ideal body weight is discussed in detail in Peterson et al. [Am J Clin Nutr 2016; 103:1197-203], incorporated herein by reference.

According to specific embodiments, the conditioning further comprises in vivo anti-B cell therapy.

Without being bound by theory, the use of the anti-B cell therapy is suggested by the inventors to lower the amount of B cells in order to reduce the incidence of autoimmunity following transplantation, which may be influenced by inadequate regulation of auto-antibodies.

According to some embodiments, the anti-B cell therapy is affected by antibodies.

Such antibodies include, for example, Rituximab, Ocrelizumab, Ofatumumab, Belimumab, Obinutumumab, Epratuzumab and Ulituximab.

According to some embodiments of the invention, the antibody is an anti-CD20 antibody.

According to a specific embodiment, the anti-B cell therapy comprises Rituximab.

Rituximab is commercially available from e.g. Genentech and Roche, e.g. under the brand names e.g. Mabthera, Rixathon, Truxima, Rituxan.

According to one embodiment, the anti-B cell therapy is administered to the subject in a single dose or in several doses e.g. 2, 3, 4, 5, 6, 7, 8, 9, 10 or more doses (e.g. daily doses) prior to cell transplantation (e.g. on days −20 to −4).

According to a specific embodiment, the subject is administered a single dose of an anti-B cell therapeutic agent (e.g. Rituximab e.g. at a dose of 100-800 mg/m² ideal body weight e.g. about 375 mg/m² ideal body weight) prior to transplantation (e.g. on days −17 to −1, on days −17 to −7, or on day −15 prior to transplantation of the T cell depleted immature hematopoietic cells).

According to a specific embodiment, the pre-transplant conditioning does not comprise in vivo anti-B cell therapy.

According to a specific embodiment, the subject is not treated with Rituximab prior to transplantation.

According to specific embodiments, the conditioning does not comprise direct total body irradiation (TBI, in which the entire body is directly exposed to the ionizing radiation) or total lymphoid irradiation (TLI, in which the lymph nodes, spleen and thymus are directly exposed to the ionizing radiation).

According to specific embodiments, the conditioning comprises a T cell debulking agent, a chemotherapeutic agent and TMI.

According to specific embodiments, the conditioning comprises an anti-B cell therapy, a T cell debulking agent, a chemotherapeutic agent and TMI.

According to specific embodiments, the conditioning comprises a T cell debulking agent, a chemotherapeutic agent, TMI and spleen irradiation.

According to specific embodiments, the conditioning comprises an anti-B cell therapy, a T cell debulking agent, a chemotherapeutic agent, TMI and spleen irradiation.

According to specific embodiments, the immunosuppressive agents administered in said conditioning consist of a T cell debulking agent, a chemotherapeutic agent and TMI.

According to specific embodiments, the immunosuppressive agents administered in said conditioning consist of an anti-B cell therapy, a T cell debulking agent, a chemotherapeutic agent and TMI.

According to specific embodiments, the immunosuppressive agents administered in said conditioning consist of a T cell debulking agent, a chemotherapeutic agent, TMI and spleen irradiation.

According to specific embodiments, the immunosuppressive agents administered in said conditioning consist of an anti-B cell therapy, a T cell debulking agent, a chemotherapeutic agent, TMI and spleen irradiation.

According to specific embodiments, the conditioning comprises ATG, Fludarabine and TMI.

According to specific embodiments, the conditioning comprises Rituximab, ATG, Fludarabine and TMI.

According to specific embodiments, the conditioning comprises ATG, Fludarabine, TMI and spleen irradiation.

According to specific embodiments, the conditioning comprises Rituximab, ATG, Fludarabine TMI and spleen irradiation.

According to specific embodiments, the immunosuppressive agents administered in said conditioning consist of ATG, Fludarabine and TMI.

According to specific embodiments, the immunosuppressive agents administered in said conditioning consist of Rituximab, ATG, Fludarabine and TMI.

According to specific embodiments, the immunosuppressive agents administered in said conditioning consist of ATG, Fludarabine, TMI and spleen irradiation.

According to specific embodiments, the immunosuppressive agents administered in said conditioning consist of Rituximab, ATG, Fludarabine, TMI and spleen irradiation.

According to specific embodiments, the chemotherapy, the T cell debulking agent and/or the anti-B cell therapy is administered prior to the TMI.

According to specific embodiments, the T cell debulking agent and/or the anti-B cell therapy is administered prior to the chemotherapy.

According to specific embodiments, the anti-B cell therapy is administered prior to the T cell debulking agent.

According to specific embodiments, the conditioning pre-transplant protocol comprises:
(i) a therapeutically effective amount of a T cell debulking agent (e.g. ATG at a total dose of about 6 mg/kg) administered in 3 daily doses on days −14 to −12 prior to transplantation of the T cell depleted immature hematopoietic cells;
(ii) a therapeutically effective amount of chemotherapy (e.g. Fludarabine at a total dose of about 120 mg/m²) administered in 4 daily doses on days −11 to −8 prior to transplantation of the T cell depleted immature hematopoietic cells; and
(iii) a therapeutically effective amount of TMI and optionally spleen irradiation, at a total dose of about 12Gy administered in 4 daily doses on days −7 to −4 prior to transplantation of the T cell depleted immature hematopoietic cells.

According to specific embodiments, the conditioning pre-transplant protocol comprises:
(i) a therapeutically effective amount of an anti-B cell therapy (e.g. Rituximab at a dose of about 375 mg/m² ideal body weight) administered in a single dose on day −15 prior to transplantation of the T cell depleted immature hematopoietic cells;
(ii) a therapeutically effective amount of a T cell debulking agent (e.g. ATG at a total dose of about 6 mg/kg) administered in 3 daily doses on days −14 to −12 prior to transplantation of the T cell depleted immature hematopoietic cells;
(iii) a therapeutically effective amount of chemotherapy (e.g. Fludarabine at a total dose of about 120 mg/m²) administered in 4 daily doses on days −11 to −8 prior to transplantation of the T cell depleted immature hematopoietic cells; and
(iv) a therapeutically effective amount of TMI and optionally spleen irradiation, at a total dose of about 12Gy administered in 4 daily doses on days −7 to −4 prior to transplantation of the T cell depleted immature hematopoietic cells.

The methods and uses described herein comprise transplanting a therapeutically effective amount of T cell depleted immature hematopoietic cells.

According to specific embodiments, the transplanting is effected following the conditioning.

The T cell depleted immature hematopoietic cells of some embodiments of the invention may be transplanted into the subject using any method known in the art for cell transplantation, such as but not limited to, cell infusion (e.g. I.V.) or via an intraperitoneal route.

Following transplantation of the cells into the subject according to the present teachings, it is advisable, according to standard medical practice, to monitor the growth functionality and immuno-compatibility of the cells, according to any one of various standard art techniques. For example, the cell numbers of immature hematopoietic cells can be monitored in a subject by standard blood and bone marrow tests (e.g. by FACS analysis).

As used herein the phrase “immature hematopoietic cells” refers to a hematopoietic tissue or cell preparation comprising precursor hematopoietic cells (e.g. hematopoietic stem cells). Such tissue/cell preparation includes or is derived from a biological sample, for example, bone marrow, mobilized peripheral blood (e.g. mobilization of CD34⁺ cells to enhance their concentration), cord blood (e.g. umbilical cord), fetal liver, yolk sac and/or placenta. Additionally, purified CD34⁺ cells or other hematopoietic stem cells such as CD131⁺ cells can be used in accordance with specific embodiments of the present teachings, either with or without ex-vivo expansion.

According to one embodiment, the immature hematopoietic cells comprise T cell depleted immature hematopoietic cells.

As used herein the phrase “T cell depleted immature hematopoietic cells” refers to a population of precursor hematopoietic cells which are depleted of T lymphocytes. The T cell depleted immature hematopoietic cells, may include e.g. CD34⁺, CD33⁺ and/or CD56⁺ cells. The T cell depleted immature hematopoietic cells may be depleted of CD3⁺ cells, CD2⁺ cells, CD8⁺ cells, CD4⁺ cells, α/β T cells and/or γ/δ T cells.

According to one embodiment, the immature hematopoietic cells comprise T cell depleted mobilized blood cells enriched for CD34⁺ immature hematopoietic cells.

According to an embodiment, the therapeutically effective amount of T cell depleted immature hematopoietic cells comprises at least about 0.1×10⁶ CD34⁺ cells, 0.5×10⁶ CD34⁺ cells, 1×10⁶ CD34⁺ cells, 2×10⁶ CD34⁺ cells, 3×10⁶ CD34⁺ cells, 4×10⁶ CD34⁺ cells, 5×10⁶ CD34+ cells, 6×10⁶ CD34⁺ cells, 7×10⁶ CD34⁺ cells, 8×10⁶ CD34⁺ cells, 9×10⁶ CD34⁺ cells, 10×10⁶ CD34⁺ cells, 15×10⁶ CD34⁺ cells or 20×10⁶ CD34⁺ cells per kg ideal body weight of the subject.

According to a specific embodiment, the therapeutically effective amount of T cell depleted immature hematopoietic cells comprises at least 2.5-20×10⁶ CD34⁺ cells (e.g. 5-10×10⁶ CD34+ cells) per kg ideal body weight of the subject.

According to a specific embodiment, the therapeutically effective amount of T cell depleted immature hematopoietic cells comprises at least 5×10⁶ CD34⁺ cells per kg ideal body weight of the subject.

According to a specific embodiment, the therapeutically effective amount of T cell depleted immature hematopoietic cells comprises at least 6×10⁶ CD34⁺ cells per kg ideal body weight of the subject.

According to a specific embodiment, the therapeutically effective amount of T cell depleted immature hematopoietic cells comprises at least 8×10⁶ CD34⁺ cells per kg ideal body weight of the subject.

According to a specific embodiment, the therapeutically effective amount of T cell depleted immature hematopoietic cells comprises at least 10×10⁶ CD34⁺ cells per kg ideal body weight of the subject.

According to one embodiment, the immature hematopoietic cells are depleted of CD3⁺ and/or CD19⁺ cells.

According to an embodiment, the therapeutically effective amount of T cell depleted immature hematopoietic cells comprises less than about 50×10⁵ CD3⁺ cells, 40×10⁵ CD3⁺ cells, 30×10⁵ CD3⁺ cells, 20×10⁵ CD3⁺ cells, 15×10⁵ CD3⁺ cells, 10×10⁵ CD3⁺ cells, 9×10⁵ CD3⁺ cells, 8×10⁵ CD3⁺ cells, 7×10⁵ CD3⁺ cells, 6×10⁵ CD3⁺ cells, 5×10⁵ CD3⁺ cells, 4×10⁵ CD3⁺ cells, 3×10⁵ CD3⁺ cells, 2×10⁵ CD3⁺ cells, 1×10⁵ CD3⁺ cells, 0.5×10⁵ CD3⁺ cells or 0.1×10⁵ CD3⁺ cells per kilogram ideal body weight of the subject.

According to a specific embodiment, the therapeutically effective amount of T cell depleted immature hematopoietic cells comprises less than 1-5×10⁵ CD3⁺ cells (e.g. 2-5×10⁵ CD3⁺ cells) per kilogram ideal body weight of the subject.

According to a specific embodiment, the therapeutically effective amount of T cell depleted immature hematopoietic cells comprises less than 5×10⁵ CD3⁺ cells per kilogram ideal body weight of the subject.

According to a specific embodiment, the therapeutically effective amount of T cell depleted immature hematopoietic cells comprises less than 4×10⁵ CD3⁺ cells per kilogram ideal body weight of the subject.

According to a specific embodiment, the therapeutically effective amount of T cell depleted immature hematopoietic cells comprises less than 3×10⁵ CD3⁺ cells per kilogram ideal body weight of the subject.

According to a specific embodiment, the therapeutically effective amount of T cell depleted immature hematopoietic cells comprises less than 2×10⁵ CD3⁺ cells per kilogram ideal body weight of the subject.

According to one embodiment, the immature hematopoietic cells are depleted of CD8⁺ cells.

According to an embodiment, the therapeutically effective amount of T cell depleted immature hematopoietic cells comprises less than 1×10⁴-5×10⁵ CD8⁺ cells (e.g. 0.1-4×10⁵ CD8⁺ cells, e.g. 1-3×10⁵ CD8⁺ cells) per kilogram ideal body weight of the subject.

According to an embodiment, the therapeutically effective amount of T cell depleted immature hematopoietic cells comprises less than about 50×10⁵ CD8⁺ cells, 25×10⁵ CD8⁺ cells, 15×10⁵ CD8⁺ cells, 10×10⁵ CD8⁺ cells, 9×10⁵ CD8⁺ cells, 8×10⁵ CD8⁺ cells, 7×10⁵ CD8⁺ cells, 6×10⁵ CD8⁺ cells, 5×10⁵ CD8⁺ cells, 4×10⁵ CD8⁺ cells, 3×10⁵ CD8⁺ cells, 2×10⁵ CD8⁺ cells, 1×10⁵ CD8⁺ cells, 9×10⁴ CD8⁺ cells, 8×10⁴ CD8⁺ cells, 7×10⁴ CD8⁺ cells, 6×10⁴ CD8⁺ cells, 5×10⁴ CD8⁺ cells, 4×10⁴ CD8⁺ cells, 3×10⁴ CD8⁺ cells, 2×10⁴ CD8⁺ cells or 1×10⁴ CD8⁺ cells per kilogram ideal body weight of the subject.

According to a specific embodiment, the therapeutically effective amount of T cell depleted immature hematopoietic cells comprises less than 5×10⁵ CD8⁺ cells per ideal kilogram body weight of the subject.

According to a specific embodiment, the therapeutically effective amount of T cell depleted immature hematopoietic cells comprises less than 4×10⁵ CD8⁺ cells per ideal kilogram body weight of the subject.

According to a specific embodiment, the therapeutically effective amount of T cell depleted immature hematopoietic cells comprises less than 3×10⁵ CD8⁺ cells per ideal kilogram body weight of the subject.

According to an embodiment, the therapeutically effective amount of T cell depleted immature hematopoietic cells comprises less than about 1×10⁶ CD8⁺ TCRα/β⁻ cells, 0.5×10⁶ CD8⁺ TCRα/β⁻ cells, 1×10⁵ CD8⁺ TCRα/β⁻ cells, 0.5×10⁵ CD8⁺ TCRα/β⁻ cells, 1×10⁴ CD8⁺ TCRα/β⁻ cells, 0.5×10⁴ CD8⁺ TCRα/β⁻ cells, 1×10³ CD8⁺ TCRα/β⁻ cells or 0.5×10³ CD8⁺ TCRα/β⁻ cells per kilogram ideal body weight of the subject.

According to a specific embodiment, the therapeutically effective amount of T cell depleted immature hematopoietic cells comprises less than 1×10⁵-1×10⁶ CD8⁺ TCRα/β⁻ cells per kilogram ideal body weight of the subject.

According to a specific embodiment, the therapeutically effective amount of T cell depleted immature hematopoietic cells comprises less than 1×10⁶ CD8⁺ TCRα/β⁻ cells per kilogram ideal body weight of the subject.

According to a specific embodiment, the therapeutically effective amount of T cell depleted immature hematopoietic cells comprises less than 5×10⁵ CD8⁺ TCRα/β⁻ cells per kilogram ideal body weight of the subject.

According to a specific embodiment, the therapeutically effective amount of T cell depleted immature hematopoietic cells comprises less than 1×10⁵ CD8⁺ TCRα/β⁻ cells per kilogram ideal body weight of the subject.

According to one embodiment, the immature hematopoietic cells are depleted of B cells.

According to an embodiment, the immature hematopoietic cells are depleted of B cells (CD19⁺ and/or CD20⁺ B cells).

According to an embodiment, the therapeutically effective amount of immature hematopoietic cells comprises less than about 50×10⁵ CD19⁺ and/or CD20⁺ cells, 40×10⁵ CD19⁺ and/or CD20⁺ cells, 30×10⁵ CD19⁺ and/or CD20⁺ cells, 20×10⁵ CD19⁺ and/or CD20⁺ cells, 10×10⁵ CD19⁺ and/or CD20⁺ cells, 9×10⁵ CD19⁺ and/or CD20⁺ cells, 8×10⁵ CD19⁺ and/or CD20+ cells, 7×10⁵ CD19⁺ and/or CD20⁺ cells, 6×10⁵ CD19⁺ and/or CD20⁺ cells, 5×10⁵ CD19⁺ and/or CD20⁺ cells, 4×10⁵ CD19⁺ and/or CD20⁺ cells, 3×10⁵ CD19⁺ and/or CD20⁺ cells, 2×10⁵ CD19⁺ and/or CD20⁺ cells or 1×10⁵ CD19⁺ and/or CD20⁺ cells per kilogram ideal body weight of the subject.

According to a specific embodiment, the therapeutically effective amount of immature hematopoietic cells comprises less than 1-5×10⁵ CD19⁺ and/or CD20⁺ cells (e.g. 3-5×10⁵ CD19⁺ and/or CD20⁺ cells) per kilogram ideal body weight of the subject.

According to a specific embodiment, the therapeutically effective amount of immature hematopoietic cells comprises less than 4×10⁵ CD19⁺ and/or CD20⁺ cells per kilogram ideal body weight of the subject.

According to a specific embodiment, the therapeutically effective amount of immature hematopoietic cells comprises less than 3×10⁵ CD19⁺ and/or CD20⁺ cells per kilogram ideal body weight of the subject.

According to a specific embodiment, the therapeutically effective amount of immature hematopoietic cells comprises less than 2×10⁵ CD19⁺ and/or CD20⁺ cells per kilogram ideal body weight of the subject.

According to a specific embodiment, the T cell depleted immature hematopoietic cells comprise two or more batches of cells, e.g. a first batch comprising CD34+ selected cells and a second batch comprising CD3⁺/CD19⁺-depleted cells (i.e. obtained from the same donor). It will be appreciated that these can be used concomitantly or subsequent to each other (e.g. on the same day or within e.g. about 1, 2, 3, 4, 5, 6, 7 days of each other, as discussed below).

Depletion of T cells, e.g. CD3⁺, CD2⁺, TCRα/β⁺, CD4⁺ and/or CD8⁺ cells, or B cells, e.g. CD19⁺ and/or CD20⁺ cells, may be carried out using any method known in the art, such as by eradication (e.g. killing) with specific antibodies or by affinity based purification e.g. such as by the use of magnetic cell separation techniques, FACS sorter and/or capture ELISA labeling.

Such methods are well known in the art and are further described herein and in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 1992). For example, cells can be sorted by, for example, flow cytometry or FACS. Thus, fluorescence activated cell sorting (FACS) may be used and may have varying degrees of color channels, low angle and obtuse light scattering detecting channels, and impedance channels. Any ligand-dependent separation techniques known in the art may be used in conjunction with both positive and negative separation techniques that rely on the physical properties of the cells rather than antibody affinity, including but not limited to elutriation and density gradient centrifugation.

Other methods for cell sorting include, for example, panning and separation using affinity techniques, including those techniques using solid supports such as plates, beads and columns. Thus, biological samples may be separated by “panning” with an antibody attached to a solid matrix, e.g. to a plate.

Alternatively, cells may be sorted/separated by magnetic separation techniques, and some of these methods utilize magnetic beads. Different magnetic beads are available from a number of sources, including for example, Dynal (Norway), Advanced Magnetics (Cambridge, MA, U.S.A.), Immuncon (Philadelphia, U.S.A.), Immunotec (Marseille, France), Invitrogen, Stem cell Technologies (U.S.A) and Cellpro (U.S.A). Alternatively, antibodies can be biotinylated or conjugated with digoxigenin and used in conjunction with avidin or anti-digoxigenin coated affinity columns.

According to an embodiment, different depletion/separation methods can be combined, for example, magnetic cell sorting can be combined with FACS, to increase the separation quality or to allow sorting by multiple parameters.

According to specific embodiments, the method comprises obtaining the T cell depleted immature hematopoietic cells.

According to a specific embodiment, the T cell depleted immature hematopoietic cells are obtained by a method comprising collecting mobilized PBMCs from a donor subject.

The term “peripheral blood mononuclear cells (PBMCs)” refers to a fraction of a blood sample comprising lymphocytes (including T cells, B cells, NK cells, etc.) and monocytes.

The term “mobilized PBMCs” refers to PBMCs obtained following administration of an agent inducing mobilization of stem cells from the bone marrow into the blood. Such agents are well known in the art and include, but not limited to, G-CSF, GM-SCF, plerixafor, T140 and T134.

According to one embodiment, mobilization is affected by G-CSF.

According to one embodiment, mobilization is affected by G-CSF and plerixafor.

According to a specific embodiment, the collection of mobilized PBMCs is obtained in a single collection.

According to a specific embodiment, the collection of the mobilized PBMCs is obtained in two, three, four, five or more daily collection, e.g. two daily collections (e.g. on consequent days or within a few days apart).

According to a specific embodiment, a back-up fraction of unmodified mobilized PBMCs containing at least about 0.1-5×10⁶ CD34⁺ cells, e.g. 2×10⁶ CD34⁺ cells, e.g. 1×10⁶ CD34⁺ cells per kg ideal body weight (e.g. obtained from the first collection) is set aside and cryopreserved.

According to one embodiment, enrichment of CD34⁺ expressing cells is affected by incubating the PBMCs with a CD34 binding agent.

According to a specific embodiment, the CD34 binding agent is an antibody.

According to a specific embodiment, the CD34 antibody is a monoclonal antibody.

According to a specific embodiment, the CD34 monoclonal antibody is conjugated to magnetic particles.

According to a specific embodiment, the CD34 monoclonal antibody is conjugated to super-paramagnetic particles.

According to one embodiment, the CD34⁺ labeled cells are selected by magnetic separation techniques.

According to a specific embodiment, the CD34 magnetically labeled cells (i.e. CD34⁺ expressing cells) are retained by the separation column (i.e. positive selection) and the CD34⁻ cells are removed. The CD34⁺ cells are then released from the column and collected. According to one embodiment, samples from each fraction are removed for cell count, viability and/or immunophenotyping.

According to one embodiment, the mobilized PBMCs are depleted of platelets using e.g. COBE 2991.

According to one embodiment, the post-platelet depleted PBMCs preparation of one embodiment is incubated with IVIg for 5-30 minutes e.g. 10-15 minutes. After the initial incubation the CD3⁺ and CD19⁺ binding agents are added to the cell preparation and incubated for e.g. 10-60 minutes, e.g. 30 minutes, on an orbital rotator.

According to a specific embodiment, the CD3 and/or CD19 binding agent is an antibody.

According to a specific embodiment, the CD3 and/or CD19 antibody is a monoclonal antibody.

According to a specific embodiment, the CD3 and/or CD19 monoclonal antibody is conjugated to magnetic particles.

According to a specific embodiment, the CD3 and/or CD19 monoclonal antibody is conjugated to super-paramagnetic particles.

According to one embodiment, at the end of the incubation, the cells are washed by centrifugation and the cell pellet re-suspended in buffer (e.g. COBE 2991) to remove excess reagent.

According to one embodiment, the CD3⁺/CD19⁺ labeled cells are selected by magnetic separation techniques.

According to a specific embodiment, the CD3⁺/CD19⁺ labeled cells are processed on CliniMACS® column.

According to a specific embodiment, the CD3⁺/CD19⁺ magnetically labeled cells (i.e. CD3⁺/CD19⁺ expressing cells) are retained by the separation column (i.e. negative selection) and the CD4⁻/CD56⁻/CD45RA⁻ cells are collected. According to one embodiment, the collected cells (CD3⁻/CD19⁻ cells) are washed and collected. According to one embodiment, samples from each fraction are removed for cell count, viability and/or immunophenotyping.

According to a specific embodiment, a second CD3⁺ depletion step is carried out in situations in which more than about 1×10⁵ CD3 to 5×10⁵ CD3, e.g. 1×10⁵ CD3 to 3×10⁵ CD3, e.g. 2.5×10⁵ CD3, e.g. 2×10⁵ CD3, per kg ideal body weight are present in the collected cell fraction.

Alternatively infusion of only part of the T cell depleted fraction is utilized so as to avoid infusion of more than 5×10⁵ CD3, e.g. 3×10⁵ CD3, e.g. 2×10⁵ CD3 cells/kg ideal body weight.

The T cell depleted immature hematopoietic cells may be used as fresh cells (e.g. within about 1, 2, 3, 4, 5, 6, 7, 8, 9 or 10 days, e.g. within about 3 days).

Alternatively, the cells may be cryopreserved until needed (e.g. for 1 day, 2-6 days, 1 week, 2 weeks, 1 month, 2 months, 4 months, 6 months, a year or more).

The protocols of some embodiments of the invention are typically used for non-syngeneic applications and therefore the T cells depleted immature hematopoietic cells (and/or veto non-GVHD inducing cells and/or the PBMCs, as further described hereinbelow) are typically allogeneic with respect to a recipient subject (e.g. from an allogeneic donor). Likewise, in cases in which xenogeneic applications may be beneficial, the cells used may be of a xenogeneic origin as discussed below. However, in cases in which a syngeneic applications may be beneficial, the cells used may be autologous with respect to a recipient subject (e.g. from the subject). Such determinations are well within the capability of one of skill in the art, especially in view of the disclosure provided.

Thus, the T cell depleted immature hematopoietic cells may be syngeneic or non-syngeneic with respect to a subject.

As used herein, the term “syngeneic” cells refers to cells which are essentially genetically identical with the subject or essentially all lymphocytes of the subject. Examples of syngeneic cells include cells derived from the subject (also referred to in the art as an “autologous”), from a clone of the subject, or from an identical twin of the subject.

As used herein, the term “non-syngeneic” cells refers to cells which are not essentially genetically identical with the subject or essentially all lymphocytes of the subject, such as allogeneic cells or xenogeneic cells.

According to specific embodiments, the T cell depleted immature hematopoietic cells are derived from a donor non-syngeneic to the subject.

According to specific embodiments, the non-syngeneic is allogeneic.

As used herein, the term “allogeneic” refers to cells which are derived from a donor subject who is of the same species as the recipient subject, but which is substantially non-clonal with the recipient subject. Typically, outbred, non-zygotic twin mammals of the same species are allogeneic with each other. It will be appreciated that an allogeneic cell may be HLA identical, partially HLA identical or HLA non-identical (i.e. displaying one or more disparate HLA determinant) with respect to the recipient subject. According to specific embodiments, the allogeneic donor is an HLA matched sibling, an HLA matched unrelated donor, an HLA haploidentical related donor or a donor displaying one or more disparate HLA determinants.

According to one embodiment, the donor is a human being.

As used herein, the term “xenogeneic” refers to a cell which substantially expresses antigens of a different species relative to the species of a substantial proportion of the lymphocytes of the subject. Typically, outbred mammals of different species are xenogeneic with each other.

The present invention envisages that xenogeneic cells are derived from a variety of species. Thus, according to one embodiment, the cells may be derived from any mammal. Suitable species origins for the cells comprise the major domesticated or livestock animals and primates. Such animals include, but are not limited to, porcine (e.g. pig), bovines (e.g., cow), equines (e.g., horse), ovine (e.g., goat, sheep), felines (e.g., Felis domestica), canines (e.g., Canis domestica), rodents (e.g., mouse, rat, rabbit, guinea pig, gerbil, hamster), and primates (e.g., chimpanzee, rhesus monkey, macaque monkey, marmoset). Cells of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

The methods and uses described herein comprise administering a therapeutically effective amount of cyclophosphamide.

According to specific embodiments, the administering the cyclophosphamide is effected following transplantation of the T cell depleted immature hematopoietic cells.

According to one embodiment, the present invention further contemplates administration of cyclophosphamide prior to transplantation (e.g. on days 6, 5, 4 or 3 prior to transplantation, i.e. D-6 to -3) in addition to the administration following transplantation as described herein.

According to specific embodiments, the therapeutic effective amount of cyclophosphamide comprises about 1-25 mg, 1-50 mg, 1-75 mg, 1-100 mg, 1-250 mg, 1-500 mg, 1-750 mg, 1-1000 mg, 5-50 mg, 5-75 mg, 5-100 mg, 5-250 mg, 5-500 mg, 5-750 mg, 5-1000 mg, 10-50 mg, 10-75 mg, 10-100 mg, 10-250 mg, 10-500 mg, 10-750 mg, 10-1000 mg, 25-50 mg, 25-75 mg, 25-100 mg, 25-125 mg, 25-200 mg, 25-300 mg, 25-400 mg, 25-500 mg, 25-750 mg, 25-1000 mg, 50-75 mg, 50-100 mg, 50-125 mg, 50-150 mg, 50-175 mg, 50-200 mg, 50-250 mg, 50-500 mg, 50-1000 mg, 75-100 mg, 75-125 mg, 75-150 mg, 75-250 mg, 75-500 mg, 75-1000 mg, 100-125 mg, 100-150 mg, 100-200 mg, 100-300 mg, 100-400 mg, 100-500 mg, 100-1000 mg, 125-150 mg, 125-250 mg, 125-500 mg, 125-1000 mg, 150-200 mg, 150-300 mg, 150-500 mg, 150-1000 mg, 200-300 mg, 200-400 mg, 200-500 mg, 200-750 mg, 200-1000 mg, 250-500 mg, 250-750 mg, 250-1000 mg per kilogram ideal body weight of the subject.

According to a specific embodiment, the therapeutic effective amount of cyclophosphamide is about 25-200 mg per kilogram ideal body weight of the subject.

According to one embodiment, cyclophosphamide is administered in a single dose.

According to one embodiment, cyclophosphamide is administered in multiple doses, e.g. in 2, 3, 4, 5 doses or more.

According to a specific embodiment, cyclophosphamide is administered in two doses.

The dose of each cyclophosphamide administration may comprise about 5 mg, 7.5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg or 500 mg per kilogram ideal body weight of the subject.

According to a specific embodiment, each dose of cyclophosphamide is 50 mg per kilogram ideal body weight of the subject.

According to specific embodiments, the cyclophosphamide may be administered to the subject between days 2 and 5 following transplantation of the T cell depleted immature hematopoietic cells.

According to a specific embodiment, cyclophosphamide is administered to the subject in two doses 3 and 4 days following transplantation of the T cell depleted immature hematopoietic cells.

Cyclophosphamide is commercially available from e.g. Zydus (German Remedies), Roxane Laboratories Inc-Boehringer Ingelheim, Bristol-Myers Squibb Co-Mead Johnson and Co, and Pfizer—Pharmacia & Upjohn, under the brand names of Endoxan, Cytoxan, Neosar, Procytox and Revimmune.

According to one embodiment, the subject is treated with additional supportive drugs, e.g. chemotherapy adjuvants.

According to one embodiment, the subject is treated with a dose of Mesna (e.g. 10 mg/kg intravenous piggy back (IVPB) just prior to the first dose of cyclophosphamide (e.g. 2 hours, 1 hour, 30 minutes, 15 minutes prior to the first dose of cyclophosphamide). According to one embodiment, administration of mesna is repeated every 4 hours for a total of 10 doses.

Mesna is commercially available from e.g. Baxter under the brand names of Uromitexan and Mesnex.

According to an embodiment, the subject is treated with ondansetron (or another anti-emetic) prior to each dose of Cyclophosphamide (Cy).

According to one embodiment, the subject is not treated with an immunosuppressive agent (e.g. aside from the CY and veto cells discussed herein).

According to one embodiment, the subject is treated with an immunosuppressive agent.

Examples of immunosuppressive agents include, but are not limited to, Tacrolimus (also referred to as FK-506 or fujimycin, trade names: Prograf, Advagraf, Protopic), Mycophenolate Mofetil, Mycophenolate Sodium, Prednisone, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, tramadol, rapamycin (sirolimus) and rapamycin analogs (such as CCI-779, RAD001, AP23573). These agents may be administered individually or in combination.

According to one embodiment, corticosteroids are not administered as a pretreatment to the veto cells.

According to specific embodiments, the subject is not treated chronically (e.g. for a prolonged period of time, e.g. for more than 8, 9, 10, 12, 14, 21, 25, 30, 45, 60 or 90 days e.g. 10-21 days, e.g. 10 or 14 days) with GVHD prophylaxis following the transplantation (e.g. with corticosteroids and/or with immunosuppressive agents).

According to one embodiment, in case of relapse after hematopoietic stem cell transplantation, the subject may be further treated by donor lymphocyte infusions (DLIs). For example, the subject may be administered with graded doses of T-cells as previously described by Dazzi et al [Dazzi, Szydlo et al., Blood, (2000) 96:2712-6] fully incorporated herein by reference.

The methods and uses disclosed herein comprise administering a therapeutically effective amount of veto non-graft versus host disease (GVHD) inducing cells.

According to specific embodiments, the veto non-GVHD inducing cells are administered following transplantation of the T cell depleted immature hematopoietic cells.

According to specific embodiments, the veto non-GVHD inducing cells are administered following administration of cyclophosphamide.

The veto non-GVHD inducing cells of some embodiments of the invention may be transplanted into the subject using any method known in the art for cell transplantation, such as but not limited to, cell infusion (e.g. I.V.) or via an intraperitoneal route.

The term “veto cells” relates to immune cells (e.g. donor derived T cells) which lead to inactivation of anti-donor recipient T cells upon recognition and binding to the veto cells. According to one embodiment, the inactivation results in apoptosis of the anti-donor recipient T cells. According to specific embodiments, the veto cell is a T cell.

Typically, a particular characteristic to veto cell biology is that the specificity of the veto activity is effected by unidirectional recognition of the veto cell by the responding T cell i.e. anti-donor recipient T cell directed against the veto cell which is killed upon binding to its veto target, due to exchange of signals following this interaction. Thus, for example, when the veto cell is a T cell, while the veto cell comprises its own specific TCR, the specificity of the veto activity is not determined by the TCR of the veto cell but rather on the TCR of the anti-donor recipient T cell in a TCR-independent manner.

Veto cells are also known to be tolerance inducing cells.

The phrase “tolerance inducing cells” as used herein refers to cells which provoke decreased responsiveness of the recipient's cells (e.g. recipient's T cells) when they come in contact with the donor cells as compared to the responsiveness of the recipient's cells in the absence of administered tolerance inducing cells.

The veto cells disclosed herein are non-graft versus host disease (GVHD) inducing cells.

The term “non-graft versus host disease” or “non-GVHD” as used herein refers to having substantially reduced or no graft versus host (GVH) inducing reactivity. Thus, the cells of some embodiments of the present invention do not significantly cause graft versus host disease (GVHD) as evidenced by survival, weight and overall appearance of the transplanted subject 30-120 days following transplantation. Methods of evaluating a subject for reduced GVHD are well known to one of skill in the art.

Numerous veto cells devoid of GVH reactivity have been described in the art, any of them can be used with specific embodiments of the present inventions. Such veto cells include for example the veto cells described in PCT Publication Nos. WO 2001/049243, WO 2002/102971, WO 2007/023491, WO 2010/049935, WO 2012/032526, WO 2013/035099, WO 2018/002924, WO 2017/009852, WO 2017/009853, WO2018/134824 and WO 2021/024264, the contents of which are fully incorporated herein by reference.

According to one embodiment, the veto non-GVHD inducing cells are not naturally occurring and are not a product of nature. These cells are typically produced by ex-vivo manipulation (e.g. exposure to an antigen or antigens in the presence of specific cytokines).

According to specific embodiments, the veto non-GVHD inducing cells comprise a central memory T-lymphocyte (Tcm) phenotype

The phrase “central memory T-lymphocyte (Tcm) phenotype” or “Tcm cells” as used herein refers to a subset of T cells which home to the lymph nodes. Cells having the Tem phenotype, in humans, typically comprise a CD3⁺/CD8⁺/CD62L⁺/CD45RO⁺/CD45RA⁻ signature. It will be appreciated that Tem cells may express all of the signature markers on a single cell or may express only part of the signature markers on a single cell. Determination of a cell phenotype can be carried out using any method known to one of skill in the art, such as for example, by Fluorescence-activated cell sorting (FACS) or capture ELISA labeling.

According to one embodiment, at least 20%, at least 25%, at least 30%, at least 35%, at least 40%, at least 50%, at least 55%, at least 60%, at least 65%, at least 70%, at least 75%, at least 80%, at least 85%, at least 90%, at least 95%, at least 99%, or even 100% of the veto non-GVHD inducing cells have the Tem cell signature.

According to a specific embodiment, cells having the Tem phenotype comprise about 20%, about 30%, about 40%, about 50%, about 60% or about 70% of the veto non-GVHD inducing cells.

According to a specific embodiment, about 10-20%, about 10-30%, about 10-40%, about 10-50%, about 20-30%, about 20-40%, about 30-50%, about 40-60%, about 50-70%, about 60-80%, about 70-90%, about 80-100%, or about 90-100% of the veto non-GVHD inducing cells have the Tem cell signature.

As mentioned, Tcm cells typically home to the lymph nodes following transplantation. According to some embodiments, the veto non-GVHD inducing cells of some embodiments of the present invention may home to any of the lymph nodes following transplantation, as for example, the peripheral lymph nodes and mesenteric lymph nodes. The homing nature of these cells allows them to exert their veto effect in a rapid and efficient manner.

Additionally or alternatively, the veto non-GVHD inducing cells of some embodiments of the present invention comprise anti-disease activity.

The term “anti-disease activity” refers to the function of the cells against a diseased cell. The anti-disease activity may be directly against a diseased cell, e.g. killing capability of the diseased cell. When the cell is a T cells, this activity may be due to TCR-dependent or TCR-independent killing [e.g. mediated by LFA1-I/CAM1 binding (Arditti et al., Blood (2005) 105(8):3365-71. Epub 2004 Jul. 6)]. Additionally or alternatively, the anti-disease activity may be indirect, e.g. by activation of other types of cells (e.g. CD4⁺ T cells, B cells, monocytes, macrophages, NK cells) which leads to death of the diseased cell (e.g. by killing, apoptosis, or by secretion of other factors, e.g. antibodies, cytokines, etc.).

A diseased cell may comprise, for example, a virally infected cell, a bacterial infected cell, a cancer cell [e.g. cell of a solid tumor or leukemia/lymphoma cell, also referred to herein as graft versus leukemia (GVL) activity of the cells], a cell associated with an autoimmune disease, a cell associated with an allergic response, or a cell altered due to stress, radiation or age.

Thus, according to specific embodiments, the veto non-GVHD inducing cells have an anti-viral, anti-bacterial, anti-fungal and/or anti-cancer activity.

According to a specific embodiment, the veto non-GVHD inducing cell cells have an anti-viral activity.

The term “anti-viral activity” refers to the function of the cells against a virally infected cell (e.g. a cell expressing viral antigen/s in the context of MHC-peptide complex on the cell surface). Typically the anti-viral activity results in killing of the infected cell.

According to a specific embodiment, the veto non-GVHD inducing cell cells have an anti-cancer activity.

The term “anti-cancer activity” refers to the function of the cells against a cancerous cell. Typically the anti-cancer activity results in killing of the cancerous cell. According to a specific embodiment, anti-cancer activity comprises graft versus leukemia/lymphoma (GVL) activity.

Thus, according to specific embodiments, the veto non-GVHD inducing cells specifically recognize a third-party antigen (e.g. a T cell comprising a TCR specific for a third party antigen).

A “third party antigen” refers to an antigen which is not present in healthy cells of either the donor or recipient.

Such an antigen may be for example a viral peptide, a bacterial peptide, a fungal peptide and/or a cancer/tumor peptide.

According to some embodiments, the veto non-GVHD inducing cells may be non-genetically modified cells or genetically modified cells (e.g. cells which have been genetically engineered to express or not express specific genes, markers or peptides or to secrete or not secrete specific cytokines) depending on the application needed (e.g. on the disease to be treated). Such determinations are well within the ability of one of ordinary skill in the art.

According to one embodiment, the cells express a chimeric antigen receptor (CAR) or a modified T cell receptor (TCR).

Accordingly, the cells of some embodiments of the invention may be transduced to express a TCR or a CAR.

As used herein “transduction with a TCR” refers to cloning of two chains (i.e., polypeptide chains), such as, an alpha chain of a T cell receptor (TCR), a beta chain of a TCR, a gamma chain of a TCR, a delta chain of a TCR, or a combination thereof (e.g. αβ chains or γδ chains). According to one embodiment, the TCR comprises the variable region of a TCR (e.g. α- and β-chains or γ- and δ-chains). Method of transducing cells (e.g. T cells) with a TCR (e.g. to generated TCR-T cells) are known in the art and are disclosed e.g. in Nicholson et al. Adv Hematol. 2012; 2012:404081; Wang and Rivière Cancer Gene Ther. 2015 March; 22(2):85-94); and Lamers et al., Cancer Gene Therapy (2002) 9, 613-623.

As used herein “transduction with a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing cells (e.g. T cells) with a CAR (e.g. to generate CAR-T) are known in the art and are disclosed e.g. in Davila et al. Oncoimmunology. 2012 Dec. 1; 1(9):1577-1583; Wang and Rivière Cancer Gene Ther. 2015 March; 22(2):85-94); and Maus et al. Blood. 2014 Apr. 24; 123(17):2625-35.

According to specific embodiments, the method comprises obtaining the veto non-GVHD inducing cells.

Methods of obtaining veto non-GVHD cells that can be used with specific embodiments of the invention are known in the art and disclosed e.g. in PCT Publication Nos. WO 2001/049243, WO 2002/102971, WO 2007/023491, WO 2010/049935, WO 2012/032526, WO 2013/035099, WO 2018/002924, WO 2017/009852, WO 2017/009853, WO2018/134824 and WO 2021/024264, the contents of which are fully incorporated herein by reference.

According to a specific embodiment, the veto non-GVHD cells are obtained or obtainable according to the methods described in PCT Publication No. WO 2021/024264.

According to a specific embodiments, the veto non-GVHD cells are obtained or obtainable according to the methods described in Examples 1-2 of the Examples section which follows, that serve as an integral part of the specification.

According to specific embodiments, the veto non-GVHD inducing cells are obtained or obtainable by a method comprising:
(i) contacting a first population of peripheral blood mononuclear cells (PBMCs) from a donor with an antibody capable of binding CD14⁺ expressing cells and selecting CD14⁺ expressing cells capable of maturing into antigen presenting cells;
(ii) loading said antigen presenting cells with a viral peptide a bacterial peptide, a fungal peptide and/or a cancer/tumor peptide;
(iii) treating a second population of PBMCs of the same donor as said first population of PBMCs with one or more agents capable of depleting CD4⁺, CD56⁺ and CD45RA⁺ expressing cells so as to obtain a population of cells comprising T cells enriched in (e.g. comprising at least 40%) memory T cells expressing a CD45RA⁻CD8⁺ phenotype;
(iv) contacting said population of cells comprising said T cells enriched in said memory T cells with said antigen presenting cells loaded with said viral peptides of step (ii) in the presence of IL-21 so as to allow enrichment of viral reactive memory T cells; and
(v) culturing said cells resulting from step (iv) in the presence of IL-21, IL-15 and/or IL-7 so as to allow proliferation of cells comprising said Tem phenotype, as further described hereinbelow.

According to one embodiment, a population of cells refers to a heterogeneous cell mixture.

As described hereinabove with respect to the T cell depleted immature hematopoietic cells, the veto non-GVHD inducing cells or PBMCs may be obtained from an allogeneic, xenogeneic or syngeneic donor with respect to a recipient subject.

According to specific embodiments, the veto non-GVHD inducing cells or PBMCs are non-syngeneic to said subject, as discussed in details hereinabove.

According to specific embodiments, the veto non-GVHD inducing cells or PBMCs are obtained from the same donor as the T cell depleted immature hematopoietic cells.

According to one embodiment, the PBMCs used for generation of the veto non-GVHD inducing cells are non-mobilized (i.e. unprimed) PBMCs, i.e. cells not obtained by means of using drugs to affect the movement of hematopoietic precursors (e.g., stem cells) from bone marrow into peripheral blood circulation.

According to one embodiment, the PBMCs for the generation of the veto non-GVHD cells are collected 5-15 days (e.g. 7-10 days, e.g. 7 days, e.g. 8 days) prior to the planned transplant date of the immature hematopoietic cells (i.e. Day 0), as discussed in detail hereinbelow. However, it is to be understood that PBMCs for the generation of the veto non-GVHD cells can be collected at any time prior to the planned transplant date. Such PBMCs can be stored as is, or can be treated as discussed below and then stored for future use (e.g. cryopreserved).

According to one embodiment, the PBMCs are collected from a donor subject using standard techniques. According to a specific embodiment, PBMCs are collected using leukapheresis, i.e. a process which essentially removes PBMCs from a donor subject, returning the remaining blood components to the donor subject.

According to one embodiment, the PBMC collection procedure (e.g. leukapheresis) yields e.g. 0.01-1000×10¹⁰ mononuclear cells (MNC), e.g. 0.1-100×10¹⁰, e.g. 1×10¹⁰ MNC.

According to one embodiment, the PBMC collection procedure (e.g. leukapheresis) yields a minimum of 500×10⁶ mononuclear cells (MNC), e.g. 500×10¹⁰ to 1000×10¹⁰ MNC, e.g. 1×10¹⁰ MNC.

According to one embodiment, the PBMCs are collected in a single collection procedure.

According to one embodiment, the PBMCs are collected in a two, three, four, five or more collection procedure (e.g. in order to obtain the required number of MNC).

According to one embodiment, if the PBMCs are collected (e.g. from the same donor subject) in two or more collection procedures, the PBMCs may be pooled together (e.g. for further processing) or used separately.

According to one embodiment, any of the aforementioned collections may be referred to as a batch. Alternatively, any group of collections (e.g. from the same donor subject in the context of collection over several days e.g. 1, 2, 3, 4, 5 days, e.g. 3 days) may be referred to as a batch.

According to one embodiment, any of the aforementioned collections of PBMCs may be kept in a collection tube for one or more days (e.g. e.g. 1, 2, 3, 4, 5 days, e.g. 1-2 days) prior to further processing (e.g. MNC isolation as discussed below).

According to one embodiment, mononuclear cells (MNC) are isolated from the PBMCs. Any standard technique known in the art can be used for MNC isolation from PBMCs. For example, according to one embodiment, the collected PBMCs are diluted (e.g. at 1:2) with Dulbecco's Phosphate-Buffered Saline (DPBS), e.g. without Calcium and Magnesium and e.g. supplemented with e.g. 0.6% ACD-A and 0.5% of 25% HAS)), and the MNC are isolated by ficoll density gradient separation. After ficoll density gradient separation, the MNC preparation of one embodiment is platelet washed (i.e. thrombowashed), e.g. 1-5 times, e.g. 1-3 times e.g. twice, by manual centrifugation and is resuspended with Wash Buffer (e.g. PBS with ACD-A and 0.5% of 25% HSA).

The PBMC preparation or MNC preparation of some embodiments of the invention is divided into two fractions (e.g. equal fractions). One PBMC or MNC fraction is further processed into antigen presenting cells (i.e. referred to herein as first population of PBMCs) and the second PBMC or MNC fraction (i.e. referred to herein second population of PBMCs) is enriched for CD8⁺ memory T cells.

According to one embodiment, the first population of PBMCs and the second population of PBMCs are from the same batch.

Alternatively, two PBMC preparations may be used from different PBMC collection procedures. Thus, according to one embodiment, the first population of PBMCs and the second population of PBMCs are from diverse batches.

According to one embodiment, antigen presenting cells (e.g. dendritic cells) are generated by first contacting the first population of PBMCs with an antibody capable of binding CD14⁺ expressing cells and selecting CD14⁺ expressing cells.

According to one embodiment, the antibody capable of binding CD14⁺ expressing cells is a CD14 monoclonal antibody. Such antibodies can be obtained commercially e.g. from BD Biosciences, Santa Cruz Biotechnology and R&D Systems.

Selecting CD14⁺ expressing cells using CD14 monoclonal antibodies may be carried out using any method known in the art, such as by the use of magnetic-activated cell sorting (MACS™) available from e.g. Miltenyi Biotec, FACS sorter and/or capture ELISA labeling.

According to one embodiment, different depletion/separation methods can be combined, for example, magnetic cell sorting can be combined with FACS, to increase the separation quality or to allow sorting by multiple parameters.

According to one embodiment, selection of CD14⁺ expressing cells is not affected by plastic adherence.

According to one embodiment, the CD14⁺ expressing cells are selected by magnetic separation techniques. Different magnetic beads are available from a number of sources, including for example, Dynal (Norway), Advanced Magnetics (Cambridge, MA, U.S.A.), Immuncon (Philadelphia, U.S.A.), Immunotec (Marseille, France), Invitrogen, Stem cell Technologies (U.S.A), Cellpro (U.S.A) and Miltenyi Biotec GmbH (Germany). Alternatively, antibodies can be biotinylated or conjugated with digoxigenin and used in conjunction with avidin or anti-digoxigenin coated affinity columns.

According to one embodiment, the CD14 monoclonal antibodies are conjugated to magnetic particles.

According to a specific embodiment, the magnetic particles comprise super-paramagnetic iron dextran particles.

According to a specific embodiment, the CD14 labeled cells are processed on CliniMACS® column.

According to a specific embodiment, the CD14 labeled cells are selected on SuperMACS™ II using the XS Separation Column.

According to one embodiment, the CD14 magnetically labeled cells (i.e. CD14⁺ expressing cells) are retained by the separation column (i.e. positive selection) and the CD14⁻ cells are removed. The CD14⁺ cells are then released from the column and collected. According to one embodiment, samples from each fraction are removed for cell count, viability and/or immunophenotyping.

According to one embodiment, viability is assessed by positive expression of 7AAD, i.e. 7AAD⁺ cells.

According to one embodiment, the CD14⁺ enriched cell preparation is re-suspended at a cell concentration of e.g. 1-10×10⁶ cells/ml, e.g. 3×10⁶ cells/ml, in cell culture medium (e.g. dendritic cell culture medium, e.g. CellGro/1% HSA). According to one embodiment, the cell culture medium is supplemented with cytokines and growth factors. Determination of cytokines and growth factors to be used is within the skill of a person of skill in the art. For example, the cell culture medium is supplemented with IL-4 (e.g. 200-2000 IU/mL, e.g. 1000 IU/mL) and GM-CSF (e.g. 1000-4000 IU/mL, e.g. 2000 IU/mL). The cell suspension is then seeded (e.g. in cell culture plates e.g. Cell Factory plates) and incubated for 12-36 hours, e.g. for 16-24 hours, e.g. for 24 hours, in at 37 °C, 5% CO₂.

According to one embodiment, in order to induce maturation of the CD14⁺ expressing cells into antigen presenting cells (e.g. dendritic cells), the CD14⁺ enriched cell preparation is cultured in the presence of maturation factors (e.g. dendritic cell maturation factors). Determination of maturation factors to be used is within the skill of a person of skill in the art. Thus, according to one embodiment, the seeded (e.g. in cell culture plates, e.g. Cell Factory plates) CD14⁺ enriched cells are cultured in the presence of IL-4 (e.g. 200-2000 IU/mL, e.g. 1000 IU/mL), GM-CSF (e.g. 1000-4000 IU/mL, e.g. 2000 IU/mL), LPS (e.g. 10-100 ng/mL, e.g. 40 ng/mL), and IFN-γ (e.g. 50-500 IU/mL, e.g. 200 IU/mL) for 10-24 hours, e.g. for 14-18 hours, e.g. for 16 hours, in at 37 °C, 5% CO₂.

After the culturing period, the antigen presenting cells (e.g. mature dendritic cells i.e. mDCs) are obtained from the cell culture. According to one embodiment, non-adherent cells are removed and the antigen presenting cells (i.e. adherent cells) are detached from the culture plates using any method known in the art (e.g. by adding ice-cold buffer e.g. ACD-A with 0.5% HAS and DPBS Buffer and incubation on ice or frozen gel packs for 10-60 minutes, e.g. 30 minutes). The harvested antigen presenting cells are then centrifuged, washed and re-suspended in medium.

According to one embodiment, antigen presenting cells (e.g. mDCs) are loaded with an antigen or antigens.

As used herein the phrase “loading” refers to the attachment of an antigen or antigens (e.g. peptides) to MHC peptides (e.g. MHC class I or II) presented in the peptide-MHC complex on the surface of the antigen-presenting cell (APC).

As used herein the phrase “antigen or antigens” refers to a soluble or non-soluble (such as membrane associated) molecule capable of inducing an immune response.

For example, an antigen or antigens can be whole cells (e.g. live or dead cells), cell fractions (e.g. lysed cells), cell antigens (e.g. cell surface antigens), a protein extract, a purified protein or a synthetic peptide.

According to one embodiment, the antigen or antigens comprise viral antigens.

According to one embodiment, the viral antigens are derived from at least 1, 2, 3, 4, 5, 6, 7, 8, 9, 10, 15, 20 or more different types of viruses.

According to one embodiment, the viral antigens are derived from 1-50, 1-40, 1-30, 1-25, 1-20, 1-15, 1-10, 1-9, 1-8, 1-7, 1-6, 1-5, 1-4, 1-3, 1-2, 2-50, 2-40, 2-30, 2-20, 2-10, 2-8, 2-6, 2-4, 3-50, 3-40, 3-30, 3-20, 3-10, 3-9, 3-7, 3-5, 3-4, 4-50, 4-40, 4-30, 4-20, 4-10, 4-8 or 4-6 types of viruses.

According to a specific embodiment, the viral antigens are derived from 1-20 types of viruses.

According to a specific embodiment, the viral antigens are derived from 1-10 types of viruses.

According to a specific embodiment, the viral antigens are derived from 1-4 types of viruses.

According to a specific embodiment, the viral antigens are derived from 2-10 types of viruses.

According to a specific embodiment, the viral antigens are derived from 2-4 types of viruses.

According to a specific embodiment, the viral antigens are derived from 4-20 types of viruses.

According to a specific embodiment, the viral antigens are derived from 4-10 types of viruses.

According to a specific embodiment, the viral antigens are derived from 4-8 types of viruses.

According to a specific embodiment, the viral antigens are derived from 4-6 types of viruses.

Exemplary viruses from which antigens can be derived (i.e. originated from) include, but are not limited to, Epstein-Barr virus (EBV), Adenovirus (Adv), cytomegalovirus (CMV), cold viruses, flu viruses, hepatitis A, B, and C viruses, herpes simplex, HIV, influenza, Japanese encephalitis, measles, polio, rabies, respiratory syncytial, rubella, smallpox, varicella zoster, rotavirus, West Nile virus, Polyomavirus (e.g. BK Virus), zika virus, parvovirus (e.g. parvovirus B19), varicella-zoster virus (VZV), Herpes simplex virus (HSV), severe acute respiratory syndrome (SARS), and severe acute respiratory syndrome coronavirus 2 (SARS-COV-2).

Particular examples of viruses and their respective antigens include, but are not limited to, BK Virus antigens include, but are not limited to, BKV LT; BKV (capsid VP1), BKV (capsid protein VP2), BKV (capsid protein VP2, isoporm VP3), BKV (small T antigen); Adenovirus antigens include, but are not limited to, Adv-penton or Adv-hexon; CMV antigens include, but are not limited to, envelope glycoprotein B, CMV IE-1 and CMV pp65, UL28, UL32, UL36, UL40, UL48, UL55, UL84, UL94, UL99 UL103, UL151, UL153, US 29, US 32; EBV antigens include, but are not limited to, EBV LMP2, EBV BZLF1, EBV EBNA1, EBV P18, and EBV P23; hepatitis antigens include, but are not limited to, the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens include, but are not limited to, immediate early proteins and glycoprotein D; HIV antigens include, but are not limited to, gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include, but are not limited to, hemagglutinin and neuraminidase; Japanese encephalitis viral antigens include, but are not limited to, proteins E, M-E, M-E-NS1, NS1, NS1-NS2A and 80% E; measles antigens include, but are not limited to, the measles virus fusion protein; rabies antigens include, but are not limited to, rabies glycoprotein and rabies nucleoprotein; respiratory syncytial viral antigens include, but are not limited to, the RSV fusion protein and the M2 protein; rotaviral antigens include, but are not limited to, VP7sc; rubella antigens include, but are not limited to, proteins E1 and E2; Severe acute respiratory syndrome (SARS-COV) antigens include, but are not limited to, S1, RBD, Nuclecapsid and Plpro; severe acute respiratory syndrome coronavirus 2 (SARS-COV-2) antigens include, but are not limited to, S1, S2, S1+S2 ECD, RBD, N antigen, S antigen and nuclecapsid; and varicella zoster viral antigens include, but are not limited to, gpl and gpll.

According to one embodiment, the antigen or antigens comprise viral peptides (or fragments thereof).

According to one embodiment, the viral peptides comprise 1-50, 1-40, 1-30, 1-25, 1-20, 1-15, 1-10, 1-9, 1-8, 1-7, 1-6, 1-5, 1-4, 1-3, 1-2, 2-50, 2-40, 2-30, 2-20, 2-10, 2-8, 2-6, 2-4, 3-50, 3-40, 3-30, 3-20, 3-10, 3-9, 3-7, 3-5, 3-4, 4-50, 4-40, 4-30, 4-20, 4-10, 4-8 or 4-6 viral peptides.

According to a specific embodiment, the viral peptides comprise 4-50 viral peptides (e.g. in a single formulation or in several formulations).

According to a specific embodiment, the viral peptides comprise 4-40 viral peptides (e.g. in a single formulation or in several formulations).

According to a specific embodiment, the viral peptides comprise 4-30 viral peptides (e.g. in a single formulation or in several formulations).

According to a specific embodiment, the viral peptides comprise 4-20 viral peptides (e.g. in a single formulation or in several formulations).

According to a specific embodiment, the viral peptides comprise 4-10 viral peptides (e.g. in a single formulation or in several formulations).

According to a specific embodiment, the viral peptides comprise 4-8 viral peptides (e.g. in a single formulation or in several formulations).

According to a specific embodiment, the viral peptides comprise 4-6 viral peptides (e.g. in a single formulation or in several formulations).

According to one embodiment, the viral peptides comprise at least 1, 2, 3, 4, 5, 6, 7, 8, 9, 10, 11, 12, 13, 14, 15, 16, 17, 18, 19, 20, 25, 30, 35, 40, 45 or 50 viral peptides (e.g. in a single formulation or in several formulations).

According to a specific embodiment, the viral peptides comprise 4 viral peptides (e.g. in a single formulation or in several formulations).

According to a specific embodiment, the viral peptides comprise 5 viral peptides (e.g. in a single formulation or in several formulations).

According to a specific embodiment, the viral peptides comprise 6 viral peptides (e.g. in a single formulation or in several formulations).

According to a specific embodiment, the viral peptides comprise 8 viral peptides (e.g. in a single formulation or in several formulations).

According to a specific embodiment, the viral peptides comprise 10 viral peptides (e.g. in a single formulation or in several formulations).

According to a specific embodiment, the viral peptides comprise 15 viral peptides (e.g. in a single formulation or in several formulations).

According to a specific embodiment, the viral peptides comprise 20 viral peptides (e.g. in a single formulation or in several formulations).

According to a specific embodiment, the viral peptides comprise 30 viral peptides (e.g. in a single formulation or in several formulations).

According to a specific embodiment, the viral peptides comprise 40 viral peptides (e.g. in a single formulation or in several formulations).

According to a specific embodiment, the viral peptides comprise 50 viral peptides (e.g. in a single formulation or in several formulations).

According to a specific embodiment, the viral peptides comprise peptides from a single organism (i.e. from one virus type).

According to a specific embodiment, the viral peptides comprise peptides from two or more organism (i.e. a mixture from 2, 3, 4, 5 or more virus types).

According to one embodiment, the viral peptides comprise a BK virus peptide.

According to a specific embodiment, the viral peptides comprise at least one of an Epstein-Barr virus (EBV) peptide, a cytomegalovirus (CMV) peptide, a BK Virus peptide and an Adenovirus (Adv) peptide.

According to a specific embodiment, the viral peptides comprise an Epstein-Barr virus (EBV) peptide, a cytomegalovirus (CMV) peptide, a BK Virus peptide and an Adenovirus (Adv) peptide.

According to a specific embodiment, the viral peptides comprise at least one of EBV-LMP2, EBV-BZLF1, EBV-EBNA1, EBV-BRAF1, EBV-BMLF1, EBV-GP340/350 EBNA2, EBV-EBNA3a, EBV-EBNA3b, EBV-EBNA3c, CMV-pp65, CMV-IE-1, Adv-penton, Adv-hexon, BKV LT, BKV (capsid VP1), BKV (capsid protein VP2), BKV (capsid protein VP2, isoporm VP3), and BKV (small T antigen).

According to a specific embodiment, the viral peptides comprise at least one of AdV5 Hexon, hCMV pp65, EBV select (discussed below) and BKV LT.

A dedicated software can be used to analyze antigen sequences to identify immunogenic short peptides, i.e., peptides presentable in context of major histocompatibility complex (MHC) class I or MHC class II.

According to a specific embodiment, the antigen or antigens comprise a mixture of pepmixes which are overlapping peptide libraries (e.g. 15 mers overlapping by 11 amino acids) spanning the entire protein sequence of three viruses: CMV, EBV, and Adeno (such pepmixes can be commercially bought e.g. from JPT Technologies, Berlin, Germany).

According to a specific embodiment, the viral peptides comprise “EBV select” i.e. a commercial product from Miltenyi Biotec comprising 43 MHC class 1 and class 2 restricted peptides from 13 different proteins from EBV (e.g. MACS GMP PepTivator® EBV Select, e.g. catalog no. 170-076-143). Additionally or alternatively, the viral peptides comprise “collection EBV” i.e., a commercial product from JPT have comprising a pepmix which includes peptides from 14 different EBV antigens. Additionally or alternatively, the viral peptides comprise PepMix™ BKV (capsid protein VP1), PepMix™ BKV (capsid protein VP2), PepMix™ BKV (capsid protein VP2, isoform VP3), PepMix™ BKV (large T antigen), PepMix™ BKV (small T antigen), commercially available from JPT.

According to another specific embodiment, the antigen or antigens comprise a mixture of seven pepmixes spanning EBV-LMP2, EBV-BZLF1, EBV-EBNA1, CMV-pp65, CMV-IE-1, Adv-penton and Adv-hexon at a concentration of e.g. 100 ng/peptide or 700 ng/mixture of the seven peptides.

According to one embodiment, the antigen or antigens comprise antigen or antigens of an infectious organism (e.g., bacterial, fungal organism) which typically affects immune comprised subjects, such as transplantation patients.

According to one embodiment, the antigen is a bacterial antigen, such as but not limited to, an antigen of anthrax; gram-negative bacilli, chlamydia, diphtheria, haemophilus influenza, Helicobacter pylori, malaria, Mycobacterium tuberculosis, pertussis toxin, pneumococcus, rickettsia, staphylococcus, streptococcus and tetanus.

As further particular examples of bacterial antigens, anthrax antigens include, but are not limited to, anthrax protective antigen; gram-negative bacilli antigens include, but are not limited to, lipopolysaccharides; haemophilus influenza antigens include, but are not limited to, capsular polysaccharides; diphtheria antigens include, but are not limited to, diphtheria toxin; Mycobacterium tuberculosis antigens include, but are not limited to, mycolic acid, heat shock protein 65 (HSP65), the 30 kDa major secreted protein and antigen 85A; pertussis toxin antigens include, but are not limited to, hemagglutinin, pertactin, FIM2, FIM3 and adenylate cyclase; pneumococcal antigens include, but are not limited to, pneumolysin and pneumococcal capsular polysaccharides; rickettsia antigens include, but are not limited to, rompA; streptococcal antigens include, but are not limited to, M proteins; and tetanus antigens include, but are not limited to, tetanus toxin.

According to one embodiment, the antigen is a superbug antigen (e.g. multi-drug resistant bacteria). Examples of superbugs include, but are not limited to, Enterococcus faecium, Clostridium difficile, Acinetobacter baumannii, Pseudomonas aeruginosa, and Enterobacteriaceae (including Escherichia coli, Klebsiella pneumoniae, Enterobacter spp.).

According to one embodiment, the antigen is a fungal antigen. Examples of fungi include, but are not limited to, candida, coccidiodes, cryptococcus, histoplasma, leishmania, plasmodium, protozoa, parasites, schistosomae, tinea, toxoplasma, and Trypanosoma cruzi.

As further particular examples of fungal antigens, coccidiodes antigens include, but are not limited to, spherule antigens; cryptococcal antigens include, but are not limited to, capsular polysaccharides; histoplasma antigens include, but are not limited to, heat shock protein 60 (HSP60); leishmania antigens include, but are not limited to, gp63 and lipophosphoglycan; Plasmodium falciparum antigens include, but are not limited to, merozoite surface antigens, sporozoite surface antigens, circumsporozoite antigens, gametocyte/gamete surface antigens, protozoal and other parasitic antigens including the blood-stage antigen pf 155/RESA; schistosomae antigens include, but are not limited to, glutathione-S-transferase and paramyosin; tinea fungal antigens include, but are not limited to, trichophytin; toxoplasma antigens include, but are not limited to, SAG-1 and p30; and Trypanosoma cruzi antigens include, but are not limited to, the 75-77 kDa antigen and the 56 kDa antigen.

According to one embodiment, the antigen or antigens comprise antigens associated with a malignant disease (e.g. cancer or tumor antigens).

According to one embodiment, the antigen is an antigen (or part thereof, e.g. antigen epitope) expressed by cancerous cells. According to one embodiment, the antigen (or part thereof) is derived from a protein expressed in a hematopoietic tissue (e.g. hematopoietic malignancy such as leukemia antigen) or expressed in a solid tumor (e.g. melanoma, pancreatic cancer, liver cancer, gastrointestinal cancer, etc.).

Examples of cancer/tumor antigens include, but are not limited to, A33, BAGE, Bcl-2, B cell maturation antigen (BCMA), BCR-ABL, β-catenin, cancer testis antigens (CTA e.g. MAGE-1, MAGE-A2/A3 and NY-ESO-1), CA 125, CA 19-9, CA 50, CA 27.29 (BR 27.29), CA 15-3, CD5, CD19, CD20, CD21, CD22, CD33, CD37, CD45, CD123, CEA, c-Met, CS-1, cyclin B1, DAGE, EBNA, EGFR, ELA2, ephrinB2, estrogen receptor, FAP, ferritin, folate-binding protein, GAGE, G250/CA IX, GD-2, GM2, gp75, gp100 (Pmel 17), HA-1, HA-2, HER-2/neu, HM1.24, HPV E6, HPV E7, hTERT, Ki-67, LRP, mesothelin, mucin-like cancer-associated antigen (MCA), MUC1, p53, PR1, PRAME, PRTN3, RHAMM (CD168), WT-1. Further cancer or tumor antigens are provided in Molldrem J. Biology of Blood and Marrow Transplantation (2006) 12:13-18; Alatrash G. and Molldrem J., Expert Rev Hematol. (2011) 4(1):37-50; Renkvist et al., Cancer Immunol Immunother (2001) 50:3-15; van der Bruggen P, Stroobant V, Vigneron N, Van den Eynde B. Peptide database: T cell-defined tumor antigens. Cancer Immun (2013), www(dot)cancerimmunity(dot)org/peptide/; Rittenhouse, Manderino, and Hass, Laboratory Medicine (1985) 16(9) 556-560; all of which are incorporated herein by reference.

According to one embodiment, the antigen or antigens comprise a mixture of antigens (e.g. a mixture of antigens of one group of antigens as discussed, e.g. viral antigens; or a mixture of antigens from different groups of antigens, e.g. viral and bacterial antigens, viral and cancer/tumor antigens).

According to one embodiment, the antigen or antigens comprise a mixture of viral peptides and cancer/tumor peptides (e.g. in a single formulation or in several formulations).

According to one embodiment, the antigen or antigens comprise a mixture of viral peptides and bacterial peptides (e.g. in a single formulation or in several formulations).

According to one embodiment, the antigen or antigens comprise a mixture of viral peptides and fungal peptides (e.g. in a single formulation or in several formulations).

According to one embodiment, loading of antigen presenting cells (e.g. mDCs) with an antigen or antigens can be carried out using any method known in the art.

According to one embodiment, in order to load (e.g. present) the viral peptides on antigen presenting cells (e.g. mDCs), the viral peptides are co-cultured with the antigen presenting cells (e.g. mDCs) for 30 minutes to 3 hours (e.g. 1 hour) at 37 °C at 5% CO₂. For instance, antigen presenting cells (e.g. mDCs) may be loaded with peptivators (e.g. AdV5 Hexon, HCMV pp65, EBV select and BKV LT) by incubation for 30 minutes to 3 hours (e.g. 1 hour) at 37 °C at 5% CO₂.

Following incubation, the viral peptide loaded antigen presenting cells (e.g. mDCs) are washed and centrifuged with e.g. ACD-A with 0.5% of 25% HAS and DPBS Buffer, and are re-suspended in cell growth medium (e.g. T cell growth medium).

According to one embodiment, the antigen or antigens (e.g. viral peptide) loaded antigen presenting cells (e.g. mDCs) are irradiated via X-Ray source. Thus, according to one embodiment, the loaded antigen presenting cells (e.g. mDCs) are irradiated with about 1-5 Gy, about 5-10 Gy, about 10-20 Gy, about 10-30 Gy, about 10-40 Gy, about 10-50 Gy, about 20-30 Gy, about 20-40 Gy, about 20-50 Gy. According to a specific embodiment, the DCs are irradiated with about 10-40 Gy (e.g. 25-30 Gy e.g. 30 Gy).

Once irradiation is complete the loaded antigen presenting cells (e.g. viral peptide loaded mDCs are washed, centrifuged and resuspended in cell growth medium (e.g. T cell growth medium).

The antigen-loaded antigen presenting cells (e.g. mDCs) are then ready to use for generation of Tcm cells from the population of cells comprising memory CD8 T cells according to some embodiments of the invention.

According to a specific embodiment, the antigen presenting cells comprise dendritic cells (DCs).

According to a specific embodiment, the antigen presenting cells comprise mature dendritic cells (mDC).

According to a specific embodiment, at least 50%, 60%, 70%, 80%, 90%, 95%, 99% or 100% of the antigen presenting cells comprise mature dendritic cells (mDC).

According to a specific embodiment, the antigen presenting cells comprise irradiated dendritic cells.

According to one embodiment, the antigen presenting cells are of the same donor subject as the veto non-GVHD inducing cells and/or the immature hematopoietic cells.

It will be appreciated that antigen presenting cells may express all of the antigens on a single cell or may express only part of the antigens on a single cell. Moreover, different antigen presenting cells (e.g. in the same preparation) may express different antigens. Accordingly, the antigen presenting cells (e.g. mDC) comprise a heterogeneous cell mixture.

According to some embodiments of the invention, the antigen or antigens (e.g. viral peptides) can be presented by genetically modified antigen presenting cells or artificial antigen presenting cells exhibiting MHC antigens (also referred to as human leukocyte antigen (HLA)) recognizable by T cells (e.g. cell line transfected with the antigen or antigens). Additionally or alternatively, antigen or antigens (e.g. viral peptides) of some embodiments of the invention can be displayed on an artificial vehicle (e.g. liposome).

Thus, antigen presenting cells (as discussed above), cell lines, artificial vehicles (such as a liposome) or artificial antigen presenting cells (e.g. leukemic or fibroblast cell line transfected with the antigen or antigens), can be used to present short synthetic peptides fused or loaded thereto or to present protein extracts or purified proteins. Such short peptides, protein extracts or purified proteins may be viral-, bacterial-, fungal-, or cancer/tumor-antigen derived peptides or peptides representing any other antigen.

As mentioned above, the method of some embodiments of the invention is affected by providing a population of cells comprising T cells, wherein the T cells in the population of cells are enriched for memory T cells expressing a CD45RA⁻CD8⁺ phenotype and depleted of CD4⁺, CD56⁺ and CD45RA⁺ expressing cells.

The term “population of cells comprising T cells” refers to a heterogeneous mixture of PBMCs comprising T cells, B cells and myeloid cells. The population of cells comprising T cells typically comprises T cells having numerous signatures, functions and capable of binding various antigens (e.g. cytotoxic T cells, memory T cells, effector T cells etc.).

According to one embodiment, the population of cells comprising T cells does not comprise erythrocytes and granulocytes.

The term “memory T cells” as used herein refers to a subset of T lymphocytes which have previously encountered and responded to an antigen, also referred to as antigen experienced T cells.

According to one embodiment, the memory T cells comprise at least about 20%, at least about 30%, at least about 40%, at least about 50%, at least about 60%, at least about 70%, at least about 80%, at least about 90%, at least about 95%, at least about 99%, or even 100% of the T cells in the population of cells.

It will be appreciated that under normal conditions (i.e. as determined in a healthy subject), the level of memory T cells comprises less than 20% of the total number of cells in a population of cells comprising T cells.

According to one embodiment, the memory T cells comprise T cells expressing a CD8 marker (i.e. CD8⁺ T cells).

According to another embodiment, the memory T cells comprise a CD8⁺CD45RO⁺ phenotype.

According to another embodiment, the memory T cells comprise a CD8⁺CD45RA⁻ phenotype.

According to another embodiment, the memory T cells comprise a CD8⁺CD45RO⁺CD45RA⁻ phenotype.

According to one embodiment, the memory T cells are devoid of CD45RA⁺ cells.

According to one embodiment, the memory T cells are devoid of CD4⁺ and/or CD56⁺ cells.

Selection of memory CD8⁺ T cells may be affected by selection of cells co-expressing CD8⁺ and CD45RA⁻ and/or cells co-expressing CD8⁺ and CD45RO⁺ and may be carried out using any method known in the art, such as by affinity based purification (e.g. such as by the use of MACS beads, FACS sorter and/or capture ELISA labeling).

Selection of memory CD8⁺ T cells may be further affected by selection of effector T cells and central memory T cells, the latter expressing e.g. CD62L, CCR7, CD27 and/or CD28.

In order to obtain a population of cells in which the T cell population comprises a high purity of memory T cells (e.g. at least about 30-50% memory T cells, e.g. 40% memory T cells) or in order to increase the number of memory T cells, PBMCs may be depleted of naïve cells, e.g. CD45RA⁺ cells, of CD4⁺ cells (e.g. T helper cells), of CD56⁺ cells (e.g. NK cells) or any other cells not comprising a memory T cell phenotype.

Depletion of naïve T cells (e.g. expressing CD45RA⁺ cells), CD4⁺ and/or CD56+ cells may be carried out using any method known in the art, such as by affinity based purification (e.g. such as by the use of MACS beads, FACS sorter and/or capture ELISA labeling).

According to one embodiment, memory T cells are obtained from peripheral blood mononuclear cells (PBMCs).

According to one embodiment, memory T cells are obtained by a method comprising treating a second population of PBMCs of the same donor subject as the first population of PBMCs with one or more agents capable of depleting CD4⁺, CD56⁺ and CD45RA⁺ expressing cells so as to obtain a population of cells comprising T cells enriched in (e.g. comprises at least 40%) memory T cells expressing a CD45RA⁻CD8⁺ phenotype.

According to one embodiment, the population of cells further comprises B cells and myeloid cells.

According to one embodiment, the CD14″ cells collected from the first population of cells are combined with the second population of cells prior to enrichment of memory T cells.

According to one embodiment, prior to enrichment of memory T cells, the CD14⁻ cells obtained from the first population of cells and/or the PBMCs obtained from the second population of cells are centrifuged and re-suspended at a concentration of e.g. 10-50×10⁶ cells/ml, e.g. 30×10⁶ cells/ml, in cell growth media e.g. T Cell Growth Media (e.g. Click's Media with advanced RPMI 1640 supplemented with 1:100 Glutamaxe and 5% Human AB Serum) along with IL-7 (30 IU/mL)). According to one embodiment, the cell growth media is supplemented with IL-7 (e.g. at a concentration of e.g. 1-100 IU/mL, e.g. 30 IU/mL). The cell suspension is then seeded (e.g. in tissue culture flasks) and incubated for 12-36 hours, e.g. for 16-24 hours, e.g. for 24 hours, in at 37 °C, 5% CO₂.

According to one embodiment, the second population of PBMCs are centrifuged and re-suspended in buffer (e.g. in CliniMACS®/0.5% HSA Buffer) to a minimum of 1:2 ratio.

According to one embodiment, the second population of PBMCs are platelet washed (e.g. thrombowash), centrifuged and re-suspended in buffer (e.g. CliniMACS®/0.5% HSA buffer).

According to one embodiment, the post-platelet depleted cell preparation of one embodiment is incubated with IVIg for 5-30 minutes e.g. 10-15 minutes. After the initial incubation the CD4⁺, CD56⁺ and CD45RA⁺ binding agents are added to the cell preparation and incubated for e.g. 10-60 minutes, e.g. 30 minutes, on an orbital rotator.

According to one embodiment, the CD4⁺, CD56⁺ and/or CD45RA⁺ binding agent is an antibody.

According to a specific embodiment, the CD4⁺, CD56⁺ and/or CD45RA⁺ binding agent is a monoclonal antibody.

According to a specific embodiment, the CD4⁺, CD56⁺ and/or CD45RA⁺ monoclonal antibody is conjugated to magnetic particles.

According to a specific embodiment, the CD4⁺, CD56⁺ and/or CD45RA⁺ monoclonal antibody is conjugated to super-paramagnetic particles.

According to one embodiment, at the end of the incubation, the cells are washed by centrifugation and the cell pellet re-suspended in buffer (e.g. CliniMACS®/0.5% HSA buffer) to remove excess reagent.

According to one embodiment, the CD4⁺/CD56⁺/CD45RA⁺ labeled cells are selected by magnetic separation techniques.

According to a specific embodiment, the CD4⁺/CD56⁺/CD45RA⁺ labeled cells are processed on CliniMACS® column.

According to one embodiment, the CD4⁺/CD56⁺/CD45RA⁺ magnetically labeled cells (i.e. CD4⁺/CD56⁺/CD45RA⁺ expressing cells) are retained by the separation column (i.e. negative selection) and the CD4⁻/CD56⁻/CD45RA⁻ cells are collected. According to one embodiment, the collected cells are washed and re-suspended in T cell Growth Medium. According to one embodiment, samples from each fraction are removed for cell count, viability and/or immunophenotyping.

According to one embodiment, the collected CD4⁻/CD56⁻/CD45RA⁻ cell fraction is adjusted at 0.01-10×10⁶ cells/ml, e.g. 2×10⁶ cells/ml, in T Cell Growth Media supplemented with cytokines and growth factors. Determination of cytokines and growth factors to be used is within the skill of a person of skill in the art. For example, the T Cell Growth Media is supplemented with IL-7 (e.g. at a concentration of e.g. 1-100 IU/mL, e.g. 30 IU/mL). The cell suspension is then seeded (e.g. in G-Rex®100) and incubated for 12-36 hours, e.g. for 16-24 hours, e.g. for 24 hours, in at 37 °C, 5% CO₂.

In order to deplete alloreactive clones from the memory T cell pool by way of antigen (e.g. viral antigen) activation, the cells comprising the memory T cells of some embodiments of the invention are contacted with the antigenic peptides, e.g. viral peptides.

According to specific embodiments, the veto non-GVHD inducing cells of some embodiments of the present invention are generated by contacting a population of cells comprising memory T cells with antigen presenting cells loaded with antigenic peptides e.g. viral peptides (such as described above) in a culture supplemented with IL-21 (e.g. in an otherwise cytokine-free culture i.e., without the addition of any additional cytokines). This step is typically carried out for about 12-24 hours, about 12-36 hours, about 12-72 hours, 12-96 hours, 12-120 hours, about 24-36 hours, about 24-48 hours, about 24-72 hours, about 36-48 hours, about 36-72 hours, about 48-72 hours, about 48-96 hours, about 48-120 hours, 0.5-1 days, 0.5-2 days, 0.5-3 days, 0.5-5 days, 1-2 days, 1-3 days, 1-5 days, 1-7 days, 1-10 days, 2-3 days, 2-4 days, 2-5 days, 2-6 days, 2-8 days, 3-4 days, 3-5 days, 3-7 days, 4-5 days, 4-8 days, 5-7 days, 6-8 days or 8-10 days and allows enrichment of antigen (e.g. viral antigen) reactive cells.

According to a specific embodiment, contacting a population of PBMC depleted of CD4+, CD56+ and CD45RA+ cells and comprising memory CD8⁺ T cells (e.g. a population of cells comprising T cells, wherein the T cells in the population of cells comprise at least 40% memory T cells), with an antigen or antigens (such as described above) in a culture supplemented with IL-21 (otherwise cytokine-free culture) is affected for 12 hours-6 days (e.g. 3 days).

Contacting a population of cells comprising memory CD8⁺ T cells with an antigen or antigens (such as described above) in a culture supplemented with IL-21 is typically carried out in the presence of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-500 IU/ml (e.g. 100 IU/ml).

According to a specific embodiment, contacting a population of cells comprising memory CD8⁺ T cells with an antigen or antigens is affected in the absence of an exogenous cytokine other than IL-21, such a culture condition enables survival and enrichment of only those cells which undergo stimulation and activation by the antigen or antigens (i.e. of antigen reactive cells, e.g. viral reactive memory T cells) as these cells secrete cytokines (e.g. IL-2) which enable their survival (all the rest of the cells die under these culture conditions).

According to one embodiment, the ratio of the population of cells comprising memory CD8⁺ T cells (i.e. CD4⁻CD56⁻CD45RA⁻ cells) to antigenic (e.g. viral) peptide loaded antigen presenting cells (e.g. mDCs) is about 2:1 to about 10:1, such as about 2:1, about 3:1, about 4:1, about 5:1, about 6:1, about 8:1 or about 10:1.

According to a specific embodiment, the ratio of the population of cells comprising memory CD8⁺ T cells (i.e. CD4⁻CD56⁻CD45RA⁻ cells) to antigenic (e.g. viral) peptide loaded antigen presenting cells (e.g. mDCs) is about 2:1 to 8:1, e.g. about 5:1.

According to a specific embodiment, the population of cells comprising memory CD8⁺ T cells (i.e. CD4⁻CD56⁻CD45RA⁻ cells) are seeded (e.g. in G-Rex®100) at a concentration of 0.01-10×10⁶ cells/ml, e.g. 1×10⁶ cells/ml, together with the viral peptide loaded antigen presenting cells (e.g. mDCs) at a ratio of about 2:1 to about 8:1, e.g. about 5:1 (memory CD8⁺ T cells: antigen presenting cells (e.g. mDC)) in T Cell Growth Media along with IL-21 (e.g. at a concentration of 50-500 IU/ml, e.g. 100 IU/ml) for 1-5 days (e.g. 3 days) in 37 °C, 5% CO₂.

According to specific embodiments, following culture with IL-21, the resultant population of cells comprising memory CD8⁺ T cells are cultured in the presence of IL-21, IL-15 and/or IL-7 so as to allow proliferation of the obtained veto non-GVHD inducing cells. This step is typically carried out for about 12-24 hours, about 12-36 hours, about 12-72 hours, about 12-96 hours, about 12-120 hours, about 12-240 hours, 24-36 hours, 24-48 hours, about 24-72 hours, 24-96 hours, 24-120 hours, 24-240 hours, about 48-72 hours, about 48-120 hours, about 48-240 hours, about 96-240 hours, about 120-144 hours, about 120-240 hours, about 144-240 hours, 0.5-1 days, 0.5-2 days, 0.5-3 days, 0.5-5 days, 0.5-10 days, 1-2 days, 1-3 days, 1-4 days, 1-6 days, 1-8 days, 1-9 days, 1-10 days, 2-3 days, 2-4 days, 2-5 days, 2-6 days, 2-8 days, 2-9 days, 2-10 days, 4-5 days, 4-6 days, 4-8 days, 4-9 days, 4-10 days, 5-6 days, 5-7 days, 5-8 days, 5-9 days, 5-10 days, 5-15 days, 6-7 days, 6-8 days, 6-9 days, 6-10 days, 6-12 days, 7-8 days, 7-9 days, 7-11 days, 7-13 days, 7-15 days, 8-9 days, 8-10 days, 9-10 days, 9-12 days, 9-15 days, 10-12 days, 10-15 days, 12-15 days, 14-16 days, 14-18 days, 16-18 days or 18-20 days. According to a specific embodiment, the resultant population of cells comprising memory CD8⁺ T cells (i.e. after culture with IL-21) are cultured in the presence of IL-21, IL-15 and IL-7 for about 6-12 days (e.g. 9 days).

This step is typically carried out in the presence of IL-21 at a concentration of about 0.001-3000 IU/ml, 0.01-3000 IU/ml, 0.1-3000 IU/ml, 1-3000 IU/ml, 10-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 250-1000 IU/ml, 500-1000 IU/ml, 750-1000 IU/ml, 10-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-21. According to a specific embodiment, the concentration of IL-21 is 50-500 IU/ml (e.g. 100 IU/ml).

This step is further carried out in the presence of IL-15 at a concentration of about 0.001-3000 IU/ml, 0.01-3000 IU/ml, 0.1-3000 IU/ml, 1-3000 IU/ml, 10-3000 IU/ml, 100-3000 IU/ml, 125-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 100-1000 IU/ml, 125-1000 IU/ml, 250-1000 IU/ml, 500-1000 IU/ml, 750-1000 IU/ml, 10-500 IU/ml, 50-500 IU/ml, 100-500 IU/ml, 125-500 IU/ml, 250-500 IU/ml, 250-500 IU/ml, 125-250 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-15. According to a specific embodiment the concentration of IL-15 is 50-500 IU/ml (e.g. 125 IU/ml).

This step is further carried out in the presence of IL-7 at a concentration of about 0.001-3000 IU/ml, 0.01-3000 IU/ml, 0.1-3000 IU/ml, 1-3000 IU/ml, 10-3000 IU/ml, 30-3000 IU/ml, 100-3000 IU/ml, 1000-3000 IU/ml, 0.001-1000 IU/ml, 0.01-1000 IU/ml, 0.1-1000 IU/ml, 1-1000 IU/ml, 10-1000 IU/ml, 30-1000 IU/ml, 100-1000 IU/ml, 250-1000 IU/ml, 500-1000 IU/ml, 750-1000 IU/ml, 10-500 IU/ml, 30-500 IU/ml, 50-500 IU/ml, 100-500 IU/ml, 250-500 IU/ml, 100-250 IU/ml, 0.1-100 IU/ml, 1-100 IU/ml, 10-100 IU/ml, 30-100 IU/ml, 50-100 IU/ml, 1-50 IU/ml, 10-50 IU/ml, 20-50 IU/ml, 30-50 IU/ml, 1-30 IU/ml, 10-30 IU/ml, 20-30 IU/ml, 10-20 IU/ml, 0.1-10 IU/ml, or 1-10 IU/ml IL-7. According to a specific embodiment the concentration of IL-7 is 1-100 IU/ml (30 IU/ml).

According to a specific embodiment, following the step of culturing with IL-21, the culture is supplemented with IL-7 (e.g. at a concentration of e.g. 1-100 IU/ml, e.g. 30 IU/mL), IL-15 (e.g. at a concentration of e.g. 50-500 IU/ml, e.g. 125 IU/mL) and IL-21 (e.g. at a concentration of 50-500 IU/ml, e.g. 100 IU/mL) at 50% of the culture volume, and cultured for about 6-12 days (e.g. 9 days) while supplementing IL-7, IL-15, IL-21 every about 48-96 hours, e.g. 48 hours, e.g. 72 hours.

According to one embodiment, the total length of culturing time for generating the cells is about 9, 10, 11, 12, 13, 14, 15, 17, 19 or 21 days (e.g. 12 days).

According to one embodiment, the cell culture is monitored for glucose levels.

According to one embodiment, the glucose is at a level comprising 10-500 mg/dl, e.g. 50-170 mg/dl.

According to one embodiment, when the glucose level is between 170 mg/dL and 130 mg/dL, cytokines IL-7, IL-15, IL-21 are added to the culture (as detailed above).

According to one embodiment, when the glucose level is between 129 mg/dL to 100 mg/dL, fresh T cell Growth medium e.g. 25% volume, e.g. 100 ml (e.g. 25% of the G-Rex®100 volume of 400 ml) plus cytokines IL-7, IL-15, IL-21 are added to the culture.

According to one embodiment, when the glucose level is between 99 mg/dL to 50 mg/dL, fresh T cell Growth medium e.g. 50% volume, e.g. 200 ml (e.g. 50% of the G-Rex®100 volume of 400 ml) plus cytokines IL-7, IL-15, IL-21 are added to the culture.

According to one embodiment, culturing further comprises adding glucose to a concentration of at least about 20 mg/dl, at least about 30 mg/dl, at least about 40 mg/dl, at least about 50 mg/dl, at least about 60 mg/dl, at least about 70 mg/dl, at least about 80 mg/dl, at least about 90 mg/dl, at least about 100 mg/dl.

According to a specific embodiment, culturing further comprises adding glucose to a concentration of at least about 50 mg/dl.

According to one embodiment, the cell culture is monitored for pH levels.

According to one embodiment, the pH is at the physiologic range (e.g. pH 7.2-7.6).

In case the pH level is not at a physiological range, the pH level may be adjusted using any method known in the art.

According to one embodiment, culturing in the presence of IL-21, IL-15 and/or IL-7 is affected in an antigen free environment (i.e. without the addition of an antigen or antigens, e.g. viral peptides). However, it is to be understood that residual antigen or antigens (e.g. viral peptides) can be present in the cell culture and thus an antigen free environment relates to a cell culture without the addition of supplementary antigen presenting cells presenting antigen or antigens (e.g. viral peptides).

According to one embodiment, in order to obtain memory CD8⁺ T cells specific to an antigen or antigens, the antigen/s (e.g. tumor antigen, viral antigen) is administered to the donor subject prior to obtaining memory CD8⁺ T cells therefrom (e.g. prior to providing the population of T cells comprising at least 40% memory CD8⁺ T cells). Any method of immunizing a cell donor against an antigen in order to elicit an immunogenic response (e.g. generation of memory CD8⁺ T cells) may be employed.

The antigen may be administered as is or as part of a composition comprising an adjuvant (e.g. Complete Freund's adjuvant (CFA) or Incomplete Freund's adjuvant (IFA)).

According to one embodiment, the antigen is administered to a donor subject once. According to one embodiment, the donor subject receives at least one additional (e.g. boost) administration of the antigen (e.g. 2, 3, 4 or more administrations). Such an additional administration may be affected 1, 3, 5, 7, 10, 12, 14, 21, 30 days or more following the first administration of the antigen.

In order to further enrich the memory CD8⁺ T cells against a particular antigen/s and to deplete alloreactive clones from the memory T cell pool, the population of cells comprising memory CD8⁺ T cells may be further contacted with the same antigen or antigens (e.g. the same antigen as administered to the cell donor), as described hereinabove.

It will be appreciated that cell samples and culture medium samples can be obtained at any stage during the process of generating the veto non-GVHD inducing cells. These can be used for evaluating cell count, cell viability, sterility, immunophenotyping, glucose and pH levels, etc. Any method known in the art can be used to implement such procedures. Non-limiting examples of such methods are described in the Examples section which follows.

According to one embodiment, the veto non-GVHD inducing cells of some embodiments of the invention can be used in conjunction with any CAR-T or TCR-T cells (e.g. CAR-T or TCR-T cells generated from cells of the veto cell donor), such as tumor specific CAR-T or TCR-T cells targeting a variety of tumor antigens. It will be appreciated that the veto cells and the CAR-T/TCR-T cells can be used concomitantly or subsequent to each other (e.g. on the same day or within e.g. about 1, 2, 3, 4, 5, 6, 7 days of each other).

The veto non-GVHD inducing cells may be used as fresh cells (e.g. within about 1, 2, 3, 4, 5, 6, 7, 8, 9 or 10 days, e.g. within about 3 days).

Alternatively, the cells may be cryopreserved until needed (e.g. for 1 week, 2 weeks, 1 month, 2 months, 4 months, 6 months, a year or more).

According to a specific embodiment, the veto non-GVHD inducing cells are administered in a single administration.

According to a specific embodiment, the veto non-GVHD inducing cells are administered in two or more administrations.

According to an embodiment, the veto non-GVHD inducing cells are administered on days 3-21 following transplantation of the T cell depleted immature hematopoietic cells, in a single or multiple administrations.

According to an embodiment, the veto non-GVHD inducing cells are administered on days 6-9 following transplantation of the T cell depleted immature hematopoietic cells, in a single or multiple administrations.

According to an embodiment, the veto non-GVHD inducing cells are administered in a single administration effected on days 6-9 following transplantation of the T cell depleted immature hematopoietic cells.

According to an embodiment, the veto non-GVHD inducing cells are administered on day 7 following transplantation of the T cell depleted immature hematopoietic cells.

According to specific embodiments, the therapeutically effective amount of the veto non-GVHD inducing cells comprises more than 1×10⁶ cells per kg ideal body weight of the subject.

According to specific embodiments, the therapeutically effective amount of the veto non-GVHD inducing cells is in the range of 0.01×10⁶ to 20×10⁶ cells per kg ideal body weight, 0.01×10⁶ to 0.5×10⁶ cells per kg ideal body weight, 0.01×10⁶ to 1×10⁶ cells per kg ideal body weight, 0.01×10⁶ to 5×10⁶ cells per kg ideal body weight, 0.1×10⁶ to 0.5×10⁶ cells per kg ideal body weight, 0.1×10⁶ to 1×10⁶ cells per kg ideal body weight, 0.1×10⁶ to 5×10⁶ cells per kg ideal body weight, 0.5×10⁶ to 1×10⁶ cells per kg ideal body weight, 0.5×10⁶ to 1×10⁶ cells per kg ideal body weight, 0.5×10⁶ to 5×10⁶ cells per kg ideal body weight, 1×10⁶ to 5×10⁶ cells per kg ideal body weight, 1×10⁶ to 20×10⁶ cells per kg ideal body weight, 5×10⁶ to 20×10⁶ cells per kg ideal body weight, 10×10⁶ to 15×10⁶ cells per kg ideal body weight, 10×10⁶ to 20×10⁶ cells per kg ideal body weight or 15×10⁶ to 20×10⁶ cells per kg ideal body weight of the subject.

According to a specific embodiment, the therapeutically effective amount of the veto non-GVHD inducing cells comprises at least 2.5×10⁶ CD8⁺ cells per kg ideal body weight of the subject.

According to a specific embodiment, the therapeutically effective amount of the veto non-GVHD inducing cells comprises at least 3×10⁶ CD8⁺ cells, at least 3.5×10⁶ CD8⁺ cells, at least 4×10⁶ CD8⁺ cells, at least 4.5×10⁶ CD8⁺ cells, at least 5×10⁶ CD8⁺ cells or at least 10×10⁶ CD8+ cells per kg ideal body weight of the subject.

According to a specific embodiment, the therapeutically effective amount of the veto non-GVHD inducing cells comprises about at least 2.5×10⁶ CD8⁺ cells per kg ideal body weight of said subject.

According to a specific embodiment, the therapeutically effective amount of the veto non-GVHD inducing cells comprises about 5×10⁶ CD8⁺ cells per kg ideal body weight of the subject.

According to a specific embodiment, the therapeutically effective amount of the veto non-GVHD inducing cells comprises about 10×10⁶ CD8⁺ cells per kg ideal body weight of the subject.

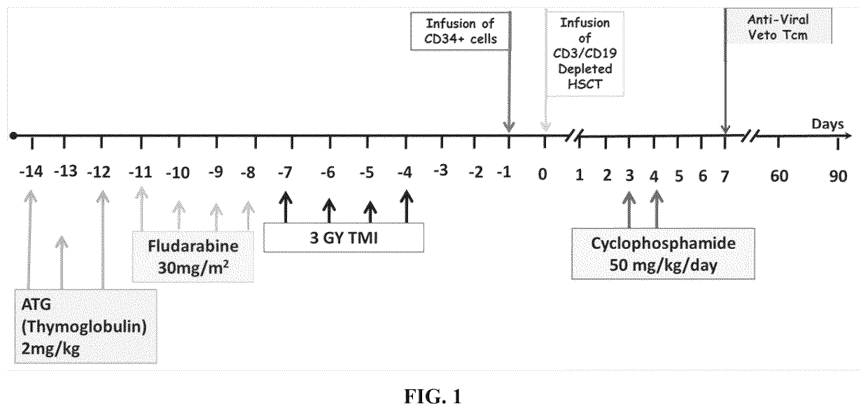

A non-limiting example of a treatment protocol that can be used with specific embodiments of the inventions is schematically provided in FIG. 1 and also described in details in Examples 1-2 of the Examples section which follows.

Hence, according to an additional or an alternative aspect of the present invention there is provided a method of treating a disease in a subject in need thereof, wherein said disease comprises pathological cells residing in a bone marrow of said subject, the method comprising:
(a) conditioning the subject under a pre-transplant conditioning protocol comprising a therapeutically effective amount of total marrow irradiation (TMI) and spleen irradiation, wherein said TMI is administered at a total dose of 12Gy administered in 4 doses on days −7 to −1 prior to transplantation of T cell depleted immature hematopoietic cells obtained from a donor non-syngeneic to said subject; and subsequently
(b) subsequent to said (a), transplanting into said subject a therapeutically effective amount of said T cell depleted immature hematopoietic cells obtained from said donor non-syngeneic to said subject, wherein said therapeutically effective amount of said T cell depleted immature hematopoietic cells comprise less than 5×10⁵ CD3⁺ T cells per kilogram ideal body weight of the subject and at least 5×10⁶ CD34⁺ cells per kilogram ideal body weight of the subject; and subsequently
(c) administering to said subject a therapeutically effective amount of cyclophosphamide wherein said therapeutically effective amount of said cyclophosphamide comprises 25-200 mg cyclophosphamide per kilogram ideal body weight of the subject, and wherein said therapeutically effective amount of said cyclophosphamide is to be administered to the subject in two doses 3 and 4 days following said transplantation of said T cell depleted immature hematopoietic cells; and subsequently
(d) administering to said subject a therapeutically effective amount of veto non-graft versus host disease (GVHD) inducing cells comprising a central memory T-lymphocyte (Tcm) phenotype and having an anti-viral activity obtained from the same donor as said T cell depleted immature hematopoietic cells, wherein said veto non-GVHD inducing cells are obtainable by:
(i) contacting a first population of peripheral blood mononuclear cells (PBMCs) from a donor with an antibody capable of binding CD14⁺ expressing cells and selecting CD14⁺ expressing cells capable of maturing into antigen presenting cells;
(ii) loading said antigen presenting cells with a viral peptide;
(iii) treating a second population of PBMCs of the same donor as said first population of PBMCs with one or more agents capable of depleting CD4⁺, CD56⁺ and CD45RA⁺ expressing cells so as to obtain a population of cells comprising T cells enriched in memory T cells expressing a CD45RA⁻CD8⁺ phenotype;
(iv) contacting said population of cells comprising said T cells enriched in memory T cells with said antigen presenting cells loaded with said viral peptides of step (ii) in the presence of IL-21 so as to allow enrichment of viral reactive memory T cells; and
(v) culturing said cells resulting from step (iv) in the presence of IL-21, IL-15 and/or IL-7 so as to allow proliferation of cells comprising said Tcm phenotype, thereby treating the disease in the subject.

According to an additional or an alternative aspect of the present invention there is provided a combination of pre-transplant conditioning protocol comprising a therapeutically effective amount of total marrow irradiation (TMI) and spleen irradiation, a therapeutically effective amount of T cell depleted immature hematopoietic cells obtained from a donor non-syngeneic to said subject, a therapeutically effective amount of cyclophosphamide and a therapeutically effective amount of veto non-graft versus host disease (GVHD) inducing cells for use in treating a disease in a subject in need thereof,
wherein said disease comprises pathological cells residing in a bone marrow of said subject,
wherein said TMI is administered at a total dose of 12Gy administered in 4 doses on days −7 to −1 prior to said T cell depleted immature hematopoietic cells,
wherein said therapeutically effective amount of said T cell depleted immature hematopoietic cells comprises less than 5×10⁵ CD3⁺ T cells per kilogram ideal body weight of said subject and at least 5×10⁶ CD34⁺ cells per kilogram ideal body weight of said subject,
wherein said therapeutically effective amount of said cyclophosphamide comprises 25-200 mg cyclophosphamide per kilogram ideal body weight of said subject to be administered in two doses 3 and 4 days following said T cell depleted immature hematopoietic cells,
wherein said veto non-graft versus host disease (GVHD) inducing cells comprise a central memory T-lymphocyte (Tcm) phenotype, have an anti-viral activity and are obtained from the same donor as said T cell depleted immature hematopoietic cells, and
wherein said veto non-GVHD inducing cells are obtainable by:
(i) contacting a first population of peripheral blood mononuclear cells (PBMCs) from a donor with an antibody capable of binding CD14⁺ expressing cells and selecting CD14⁺ expressing cells capable of maturing into antigen presenting cells;
(ii) loading said antigen presenting cells with a viral peptide;
(iii) treating a second population of PBMCs of the same donor as said first population of PBMCs with one or more agents capable of depleting CD4⁺, CD56⁺ and CD45RA⁺ expressing cells so as to obtain a population of cells comprising T cell enriched in memory T cells expressing a CD45RA⁻CD8⁺ phenotype;
(iv) contacting said population of cells comprising said T cells enriched in said memory T cells with said antigen presenting cells loaded with said viral peptides of step (ii) in the presence of IL-21 so as to allow enrichment of viral reactive memory T cells; and
(v) culturing said cells resulting from step (iv) in the presence of IL-21, IL-15 and/or IL-7 so as to allow proliferation of cells comprising said Tcm phenotype.

The number of administrations and the therapeutically effective amount of the pre-transplant and post-transplant cells, immunosuppressive drugs and/or immunosuppressive irradiation may be adjusted as needed taking into account the subject's response to the regimen. Determination of the number of administrations and the therapeutically effective amount is well within the capability of those skilled in the art, especially in light of the detailed disclosure provided herein.

The cells and drugs of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cell or drug described herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

According to one embodiment, the cells are formulated for administration as fresh cells.

According to one embodiment, the cells are formulated for administration as cryopreserved cells.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer as defined herein) or prolong the survival of the subject being treated. Determination of the therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide ample levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques.

### Example 1

### Clinical Protocol

### Patient Inclusion Criteria

Age 12-75 years
Patients with a diagnosis either acute myeloid leukemia (AML), myelodysplastic syndromes (MDS), chronic myeloid leukemia (CML) or multiple myeloma (MM).
Patients with aplastic anemia and severe immune deficiency or non-malignant bone marrow failure states. Patients with severe thalassemia requiring regular blood transfusions or sickle cell disease with severe clinical features (these include any clinically significant sickle genotype, for example, Hemoglobin SS (Hb SS), Hemoglobin SC (Hb SC), Hemoglobin S Beta thalassemia (Hb SB), or Hemoglobin S-OArab genotype] with at least one of the following manifestations:
a. Clinically significant neurologic event (stroke) or neurological deficit lasting >24 hours;
b. History of two or more episodes of acute chest syndrome (ACS) in the 2-year period preceding enrollment or referral despite adequate supportive care measures (i.e. asthma therapy);
c. An average of three or more pain crises per year in the year period preceding enrollment or referral (required intravenous pain management in the outpatient or inpatient hospital setting);
d. Administration of regular red blood cell (RBC) transfusion therapy, defined as 8 or more transfusion events per year (in the 12 months before enrollment) to prevent vaso-occlusive clinical complications (i.e. pain, stroke, or acute chest syndrome);
e. An echocardiographic finding of tricuspid valve regurgitant jet (TRJ) velocity ≥2.7 m/sec.
f. Ongoing high impact1 chronic pain on a majority of days per month for ≥6 months as defined as ONE or more of the following: Chronic pain without contributory SCD complications2, OR Mixed pain type in which chronic pain is occurring at site(s) (arms, back, chest, or abdominal pain) unrelated to any sites associated with Contributory SCD complications2 (e.g. leg ulcers and/or avascular necrosis).
Patients with hematological malignancies must have had persistent or progressive disease despite initial chemotherapy and must have achieved stable disease or a partial or complete response to their most recent chemotherapy. Patients with low bulk or indolent disease are eligible without additional treatment. Patients with intermediate or high risk acute myeloid leukemia by European LeukemiaNet (ELN) criteria in first remission are eligible.
Availability of a haploidentical related donor
Karnofsky performance status ≥70%
Adequate major organ system function as demonstrated by:
   Left ventricular ejection fraction of at least 40%
   Pulmonary function test (PFT) demonstrating an adjusted diffusion capacity of least 50% predicted value for hemoglobin concentration
   Serum creatinine≤1.5 mg/dl.
   Serum glutamic-pyruvic transaminase (SGPT)≤200 IU/ml
   Bilirubin<1.5 mg/dl (unless Gilbert's syndrome).
   Negative pregnancy test in women with child bearing potential.

### Patient Exclusion Criteria

HIV seropositive.
Uncontrolled infection or serious medical or psychiatric condition that would limit tolerance to the protocol treatment.
Active CNS malignancy.
Availability of medically eligible, human leukocyte antigen (HLA)-matched related stem cell donor.

### Treatment Plan

A schematic representation of the treatment protocol is provided in FIG. 1.

There are two collections from the donor, one “unprimed” mononuclear cells (MNC) for the preparation of the Veto Cells and the second collection of mobilized peripheral blood progenitor cells (PBPCs) for preparation of the megadose T cell depleted stem cell transplant. Both collections follow standard of care procedures (SOC) and donors sign SOC consent for donation.

The collection of unprimed (i.e. non-mobilized) peripheral blood mononuclear cells for production of the anti-viral central memory CD8⁺ veto T cells may be done at any time, but generally are done about 8 days before the planned transplant date (i.e. Day 0, i.e. DO). The veto cells are either used fresh at the end of their manufacturing and are to be infused on D+7, or are cryopreserved at the end of their manufacturing.

The collection of the mobilized PBPCs is effected using G-CSF and plerixafor, if clinically necessary, for the megadose transplant. The donor is treated with G-CSF 10 mcg/kg daily on 6 consecutive days starting on day −6 and a PBPC collection is performed on day −2 and day −1. Donors may receive plerixafor on day −3 and day −2 if clinically indicated.

An aliquot of PBPC containing ≥1×10⁶ CD34⁺ cells/kg is taken from the first or second collection and cryopreserved as a backup. The cells from first day's collection on day −2 are stored overnight and undergo CD34-selection using the CliniMacs device (Miltenyi) and infused on day-1. The second PBPC collection is done on day −1; the cells are stored overnight and are depleted of CD3+/CD19+ cells using the CliniMacs device (Miltenyi) and infused on day 0. A maximum T-cell dose in the two T-cell depleted grafts is no more than 2×10⁵ CD3+ cells/kg ideal recipient body weight.

### Consideration for Mobilization Failure

A mobilization failure is considered if the total number of CD34+ cells in the T-cell depleted graft is <5×10⁶/Kg ideal body weight, and the backup T cell replete PBPC is combined with the T cell depleted cells and infused on day 0. In this case, the patient does receive veto cells and analyzed separately.

### Production of Anti-Viral Central Memory CD8⁺ Veto T Cells (Veto Cells)

A detailed protocol for production of veto cells is provided in Example 2, hereinbelow. This section provides a brief description of some embodiments of the invention.

The protocol comprises three steps:

### Step 1: Preparation of Stimulators (Day −8 to Day −5)

Preparation of Donor DC from Monocytes in PBMC

Day −8: Approximately 1×10¹⁰ mononuclear cells are collected by leukapheresis and kept overnight.

Day −7: The cells are processed by ficoll separation and thereafter half of the mononuclear cells are used for monocyte isolation by CD14 magnetic beads (Miltenyi magnetic beads sorting system). Thereafter, the CD14⁺ monocytes are differentiated to immature DC with granulocyte macrophage-colony stimulating factor (GM-CSF) and interleukin 4 (IL-4).

In parallel, the CD14⁻ (i.e. CD14 neg) cells are combined with half of the initial mononuclear cells obtained after ficoll isolation and stored overnight in 37 °C. 5% O2/CO2 in the presence of IL-7. These cells are used for the enrichment of CD8⁺ memory T cells on the next day.

Day −6: DC Maturation by addition of cytokine-cocktail comprising lipopolysaccharide (LPS), Interferon gamma (IFNγ), GM-CSF, and IL-4 for 16 hours of incubation.

Day −5: Mature DCs (mDCs) are harvested, loaded with a viral peptide cocktail and irradiated.

Step 2: Preparation of Responders and Co-Culture with Viral Loaded mDC (Day −6 to Day −2):
A) Memory T Cell (CD4⁻CD56⁻CD45⁻) Purification (from Donor PBMC-Responders)

Day −6: Donor CD14 cells combined with half of the mononuclear cells that were incubated overnight with IL-7, are depleted of CD4⁺, CD56⁺, and CD45RA⁺ cells by magnetic beads and stored for an additional night in 37 °C. 5% 02/CO₂ in the presence of IL-7.

### B) Establishment of Responder (Donor) Stimulator (Donor Viral Peptides Loaded DCs) Co-Culture.

Days −5 to −2: Purified memory responder T cells (CD4⁻CD56⁻CD45RA⁻) are incubated for 3 days with viral peptide loaded mDCs in the presence of IL-21.

### Step 3: Differentiation and Expansion of Anti-Viral Veto Tcm (Day −2 to +7):

A) Expansion of anti-viral central memory CD8⁺ veto T cells (Tcm)

Days −5 to −2: Initiation of Tem phenotype with IL-21 only as described hereinabove.

Days −2 to +7: Expansions of Tem cultures by splitting according to glucose level consumption and addition of medium supplemented with the following cytokines: IL-7, IL-15 and IL-21 depending on the observed expansion in culture.

Day +7: Harvest and infusion of cells.

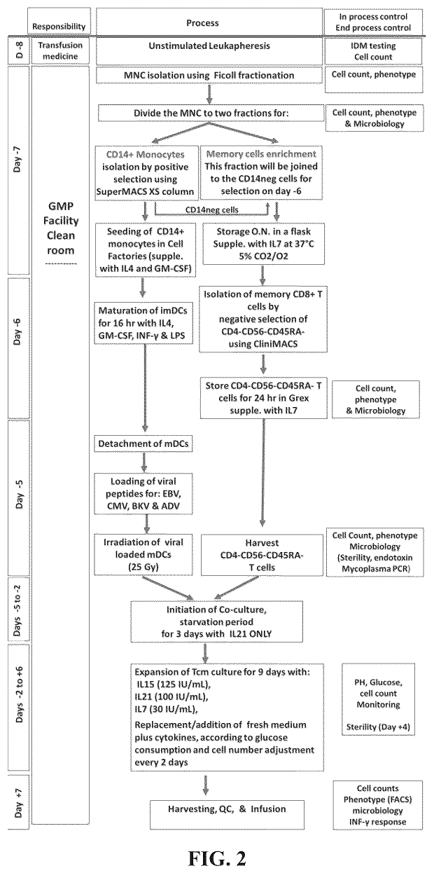

Approximate timeline of Tcm production and DC preparation from the intended PBMC donor is shown in FIG. 2. Cells are harvested and infused on day +7 post hematopoietic stem cell transplant.

An aliquot of the anti-viral central memory CD8⁺ veto cells are assessed for release criteria and functional immune studies.

### Treatment Plan-Conditioning Regimen

A schematic representation of the conditioning regimen is provided in FIG. 1.

### Day and Treatment:

−15 Admit/Intravenous (IV) hydration)
−14 ATG (Thymoglobulin) 2 mg/kg
−13 ATG (Thymoglobulin) 2 mg/kg
−12 ATG (Thymoglobulin) 2 mg/kg
−11 Fludarabine 30 mg/m2
−10 Fludarabine 30 mg/m2
−9 Fludarabine 30 mg/m2
−8 Fludarabine 30 mg/m2
−7 Total marrow radiation 3 Gy
−6 Total marrow radiation 3 Gy
−5 Total marrow radiation 3 Gy
−4 Total marrow radiation 3 Gy
−3 Rest
−2 Rest
−1 Infusion of megadose T-cell depleted PBPCs (containing the CD34 selected cells)
0 Infusion of CD3−/CD19− depleted cells
+3 Cyclophosphamide (CY) 50 mg/kg/day IV
+4 Cyclophosphamide (CY) 50 mg/kg/day IV
+7 Infusion of Anti-viral veto cells at doses as indicated.

Of note, patients receive a dose of Mesna 10 mg/kg intravenous piggy back (IVPB) just prior to the first dose of cyclophosphamide. This is repeated every 4 hours for a total of 10 doses. Patients also receive ondansetron (or other anti-emetic) prior to each dose of Cyclophosphamide (Cy).

Another set of patients is also treated with Rituximab 375 mg/m² on day −15. Without being bound by theory the administration of Rituximab is in order to reduce B cell number and prevent autoimmunity.

### Total Marrow Irradiation (TMI)

Patients receive total marrow irradiation (TMI) of 12 Gy in 4 fractions, targeting the bone marrow and also the spleen.

Radiotherapy Simulation—Patients are immobilized in the supine position using a full-body Vac-Lok bag (CIVCO Radiotherapy, Orange City, Iowa, U.S.). The arms are relaxed, down by the sides, in a non-akimbo fashion, with fingers holding onto the bag. Additional devices may be used as needed for patient comfort to ensure positioning reproducibility. Two planning CT scans are acquired—one head-first supine scan, and one feet-first supine scan. The CT scans' field of view should include the immobilization device and the slice thickness should be ≤3 mm.

Treatment Planning System—The planning CT scans are imported into the Raystation (RaySearch Laboratories, Stockholm, Sweden) treatment planning system. All contouring and radiotherapy treatment planning are performed in Raystation.

Definition of Target Volume—The target volumes are contoured on the planning CT scans. The high-dose target volumes include the whole skeleton and spleen. The Clinical Target Volume (CTV) for the bone marrow (CTV_Bone) may be defined using a threshold function over the patient's body to include all bones. The CTV for the spleen (CTV_Spleen) may be generated by manual delineation or automatic segmentation and reviewed by the treating physician. To account for patient setup uncertainty, the high-dose Planning Target Volumes (PTVHigh) are generated by applying a 3-10 mm expansion to the high-dose CTVs. The low-dose target volumes include the lymph node stations, liver, and brain (CTV_LN, CTV liver, CTV brain, respectively). CTV_LN is delineated by the treating physician. The contours for CTV liver and CTV brain may be generated by manual delineation or automatic segmentation. All contours generated using automatic segmentation are reviewed on every slice and edited as necessary. Low-dose Planning Target Volumes (PTV Loware generated by applying a 3-10 mm expansion to the low-dose CTVs. The target volumes determined by the treating physician are peer-reviewed by members of the Hematology Section in the Division of Radiation Oncology.

Definition of Organs at Risk—The organs at risk (OAR) are contoured on the planning CT scans. Contours of the lungs and kidneys are required. Additional organs at risk may be contoured for dose evaluation. These optional contours may include the bladder, breasts, esophagus, eyes, abdominal cavity, small bowel, bowel, larynx, lenses, optic nerves, oral cavity, uterus, parotids, prostate, rectum, stomach, testes, thyroid, and genitals, among others. The contours may be generated by manual delineation or automatic segmentation. If any of the contours are generated using automatic segmentation, the contours must be reviewed on every slice and edits made as necessary. All organs at risk are labeled following the American Association of Physicists in Medicine (AAPM) Task Group 263 (TG-263) nomenclature. For instance, for the required organs at risk, the labels will be “Lung_R”, “Lung_L”, “Lungs”, “Kidney_R”, “Kidney_L”, and “Kidneys”.

Target Volume Dose Prescription—The high-dose target volumes are prescribed to 12 Gy in 4 fractions at 3 Gy/fraction. The treatment planning goals include D90%≥12 Gy (dose to 90% of the target volume should be greater than or equal to 12 Gy) and D1%<120% (dose to hottest 1% of the volume less than 120% of the prescription dose). The low-dose target volumes are evaluated to ensure the mean dose is between 3 Gy and 5 Gy.

Organs at Risk Goals—The goal mean doses to the individual lungs and kidneys shall be less than 5 Gy. While recommended limits for the lungs and kidneys are provided below, the goal for all non-target structures is to limit doses as low as reasonably achievable (ALARA) without compromising target coverage.

Dose Compliance Criteria—Radiotherapy plans are evaluated based on target volume and OAR dosimetric parameters in Table 1 hereinbelow. Plans not meeting these criteria constitute a protocol deviation and additional treatment planning is recommended.

TABLE 1
Treatment planning parameters

| | Dosimetric | Per Protocol | Acceptable |
|---|---|---|---|
| Structure | Parameter | (Gy) | Variation (Gy) |
| PTV_High | D90% | ≥12 | ≥11 |
| | D1% | ≤14.4 (120%) | ≤15.6 (130%) |
| PTV_Low | Dmean | ≥3 and ≤5 | ≥2 and ≤6 |
| Lung_R, Lung_L | Dmean | ≤5 | ≤7 |
| Kidney_R, Kidney_L | Dmean | ≤5 | ≤7 |

Radiotherapy Planning Priorities—The priority of radiotherapy planning goals, in order of importance:
1. High-dose target volumes' minimum and maximum dosimetric criteria
2. Mean lung doses
3. Mean kidney doses
4. Mean dose to the low-dose target volumes
5. Dose to all other OARs: ALARA
Radiotherapy Planning Techniques—TMI is planned with volumetric modulated arc therapy (VMAT) for the body and 3D-conformal radiation therapy (3D-CRT) for the legs. For the VMAT body plan, 6 to 12 overlapping arcs from 3 to 6 different isocenters may be used. Depending on patient height and anatomy, more arcs and isocenters may be needed for larger patients. The isocenters for these arcs and field sizes are determined based on patient anatomy to optimize the travel of the multileaf collimators. For each arc, there is ≥2 cm overlap region with the arc superior to it, and another ≥2 cm overlap region with the arc inferior to it. For the 3D legs plan, the multileaf collimators conform to the PTV from the beam's-eye-views. There is ≥2 cm overlap region between the VMAT body plan and the 3D leg plan.
   Dose calculation is performed on the planning CT scans. The calculation volume should fully enclose the patient and immobilization devices. The size of the dose grid should be ≤3 mm in all directions.
   The VMAT body plan and 3D legs plan are set to be interdependent. Once the dose distribution is calculated for the 3D legs plan, this dose distribution is set as the background dose for the VMAT body plan. Inverse optimization is then performed for the VMAT body plan to achieve the plan objectives described in the previous section.

Radiotherapy Machine—Radiation therapy is delivered with 6 megavoltage (MV) photons from a TrueBeam (Varian Medical Systems, Palo Alto, California, U.S.) linear accelerator equipped with the Millennium 120 multileaf collimator. The radiation output is calibrated following the AAPM TG-51 guidelines. Daily, monthly, and annual quality assurance of the linear accelerator is performed per state and national guidelines. Patient-specific quality assurance of all the treatment fields in the plan is performed and reviewed by a qualified medical physicist prior to the first fraction of the patient's radiation treatments.

Radiotherapy Schedule—Due to the interdependent nature of systemic therapy and radiation therapy within the patient's overall treatment regimen, all radiotherapy fractions must be delivered on the scheduled days and no treatment breaks are allowed.

Patient Setup—The patient's position during treatment is reproduced as closely as possible to the setup during the patient's simulation which serves as the reference. Daily image-guidance is used to compare the daily versus reference patient position. Image-guidance is performed using the on-board imaging system, which consists of a kilovoltage (kV) x-ray source and imaging panel, as well as the megavoltage (MV) treatment beam.

All image registrations are performed based on bony landmarks. For the VMAT body plan, the patient is in the head-first supine position. After initial positioning of the patient, a kV cone-beam CT scan is acquired for the thoracic isocenter. Translational shifts may be applied remotely by moving the couch, while rotational shifts must be applied by adjusting the patient's body directly. Orthogonal kV x-ray images is acquired for each isocenter sequentially from the head to the pelvis. Radiation therapists use a combination of automatic and/or manual registration to determine the required shifts, and adjust the patient accordingly. Following any adjustments, a final set of orthogonal kV x-ray images is acquired for each isocenter to confirm patient position prior to treatment, and sent to the treating physician for approval. Prior to the treatment of each isocenter, MV images are also acquired to ensure the correct isocenter shifts have been applied.
For the 3D legs plan, the patient is turned to the feet-first supine position. Orthogonal kV images are acquired to match the daily bony anatomy position to the reference position of the lower extremities at simulation.

### Anti-Viral Veto Cell Dose Levels-CD8⁺ Cell Dose Escalation

Premedication for the Veto cells should not include corticosteroids.
Dose level 1:2.5×10⁶ CD8⁺ cells per kg ideal body weight
Dose level 2:5.0×10⁶ CD8⁺ cells per kg ideal body weight
Dose level 3:10×10⁶ CD8⁺ cells per kg ideal body weight
No additional post-transplant immunosuppressive therapy is administered as GVHD prophylaxis.
   Consideration if manufactured cell product is less than the planned dose level

Dose level one comprises 2.5×10⁶ cells per kg ideal body weight. Of note, a lower dose is not typically considered acceptable.

For dose level 2, the target dose is 5×10⁶ cells per kg ideal body weight, however, a dose of 2.6 to 5×10⁶ cells per kg ideal body weight is considered adequate and considered dose level 2.

For dose level 3, the target dose is 10×10⁶ cells per kg ideal body weight, however, a dose of 5.1 to 10×10⁶ cells per kg ideal body weight is considered adequate, and considered dose level 3.

### Supportive Care

No post-transplant growth factors
No post-transplant immunosuppressive therapy is administered as GVHD prophylaxis.
Other aspects of supportive care are administered as clinically indicated (e.g. support by G-CSF in cases in which engraftment is slow, or treatment of GVHD by e.g. methylprednisolone, tacrolimus or other immunosuppressive medications). Concomitant medications administered to the patient during the study are documented in the patient's primary medical record.

### Donor Lymphocyte Infusion (DLI) for Persistent or Progressive Disease

If GVHD is not present, patients who have achieved engraftment and have ≥5% myeloid donor cells and who have progressive disease at any time or persistent disease at ≥3 months post-transplant may receive donor lymphocyte infusion as standard of care or alternative therapy.

Treatment of graft failure—was done as described in Example 1 of International Patent Application Publication No. WO2021/024264.

Interventions for GVHD occurring after transplant—was done as described in Example 1 of International Patent Application Publication No. WO2021/024264.

### Evaluation During Study

Effort is made to adhere to the schedule of events and all protocol requirements. Variations in schedule of events and other protocol requirements that do not affect the rights and safety of the patient are not considered as deviations. Such variations may include laboratory assessments completed outside of schedule and occasional missed required research samples. Missed samples for correlative studies are not constitute protocol deviations.

### Evaluation Prior to Transplant (Baseline):

Standard work up for transplant as well as disease assessment is done prior to study entry as part of diagnostic or routine pre-transplant evaluation. The following tests are standard of care pre-transplant tests and not protocol specific. The results are used to determine transplant eligibility and are not repeated prior to the beginning of treatment. If the treatment is delayed for more than 30 days after consenting, the PI or designee should determine which, if any, tests need to be repeated as clinically indicated.
CBC, differential and platelets
Bilirubin, serum creatinine and creatinine clearance, ALT, albumin, electrolytes, LDH, alkaline phosphatase
Infectious diseases panel (hepatitis serology (B, C), HIV, HTLV, I/II, CMV, TPHA screen), toxoplasma serology, Strongyloides serology (if indicated), tuberculosis, IRGA (if indicated)
PT and PTT
ABO and Rh typing
Bone marrow aspiration with cytogenetics and molecular studies if clinically indicated
Chimerism analysis baseline
Pregnancy test in females of childbearing potential
Pulmonary function test with DLCO
Echocardiogram to assess Left ventricular EF and pulmonary artery pressure
Chest X ray and ECG
Serum for donor-specific anti-HLA antibodies (DSA)

The active treatment period for patients treated in this study is from the beginning of the preparative regimen through Day +42 post-transplant. After that, patients have follow-up as clinically indicated through D+100. Thereafter, disease relapse, infections, acute and chronic GVHD and survival data are collected according to the standard follow up of stem cell transplant recipients. After one year, patients are removed from the study. Acute GVHD and Chronic GVHD are scored according to NIH Consensus Criteria.

### Standard Post Evaluations

Standard Post Evaluations are per SOC post allogeneic transplant. Bone marrow aspiration and peripheral blood—to evaluate treatment response, engraftment, chimerism and immune reconstitution—are performed monthly for 3 months and as clinically indicated.

The development of acute GVHD is assessed and scored by NIH consensus criteria. Disease relapse or progression is assessed by morphologic relapse of the patient's malignancy. Data on bacterial, viral fungal and parasitic infections is collected. The development of chronic GVHD is described as per NIH Consensus Criteria [Jagasia M H, et al. National Institutes of Health Consensus Development Project on Criteria for Clinical Trials in Chronic Graft-versus-Host Disease: I. The 2014 Diagnosis and Staging Working Group report. Biol Blood Marrow Transplant. (2015) 21:389-401 e381].

### Table of Assessments

TABLE 2
Table of Assessments

| | Pretreatment | | | | | | | |
|---|---|---|---|---|---|---|---|---|
| | (within | | | | | | | |
| | 30 days | Week 1 | Week 2 | Week 3 | Day 30 | Day 60 | Day 90 | Day 100 |
| | of study | (+/−1 | (+/−2 | (+/−3 | (+/−5 | (+/−5 | (+/−5 | (+/−14 |
| | enrollment) | day) | days) | days) | days) | days) | days) | days) |
| Karnofsky Performance Status | X | | | | | | | |
| Laboratory Tests¹ | X | | | | | | | |
| Pregnancy Test² | X | | | | | | | |
| CBC | | X | X | X | X | X | X | X |
| Chimerism Analysis | X | X | X | X | X | X | X | X |
| Pulmonary Function Test with DLCO | X | | | | | | | |
| ECHO | X | | | | | | | |
| Chest x-ray | X | | | | | | | |
| Bone marrow aspiration and biopsy³ | X | | | | X³ | X³ | X³ | |
| Correlative Studies (participants) | X | | | | X | X | X | |
| Correlative Studies (donors) | X | | | | | | | |

¹Baseline Laboratory Tests: CBC, differential and platelets, bilirubin, serum creatinine and creatinine clearance, ALT, albumin, electrolytes, LDH, alkaline phosphatase, infectious diseases panel (hepatitis serology (B, C), HIV, HTLV, I/II, CMV, TPHA screen), toxoplasma serology, strongyloides serology (if indicated), tuberculosis IRGA (if indicated), PT and PTT, ABO and Rh typing, serum for donor-specific anti-HLA antibodies (DSA).
²Pregnancy test (urine) in females of childbearing potential.
³If clinically indicated.

### Correlative Studies

Immune reconstitution is assessed by flow cytometry immunophenotype panel and antiviral responses by tetramer analysis. Immune tolerance test is performed approximately 3 months post-transplant. Missed samples for correlative studies are not constitute protocol deviations.

### Biospecimen Sample Procurement

Total marrow irradiation may alter the immune responses of donor lymphocytes as well as myeloid cells by better preserving the function of lymphoid organs. Both circulating cytokines, immune cell phenotype and function as well as myeloid and NK cell phenotype and function are analysed.

Blood samples are taken within 1-2 weeks prior to ATG, within 4 days prior to TMI, and 2-4 weeks after cyclophosphamide/anti-viral veto Tcm. A unique identifier is given for each patient. Bone marrow aspiration and biopsy samples are obtained at Days 30, 60, and 90 during the standard of care procedure, when clinically indicated (i.e., a separate bone marrow aspiration/biopsy procedure is not performed for the purpose of obtaining samples for correlative studies). Bone marrow aspiration and biopsy samples, peripheral blood mononuclear cells and serum are obtained from patients.

Peripheral blood mononuclear cell isolation-Blood is collected in a 7-10 cc green top tube with heparin. Half of the plasma is collected and aliquoted. The remaining plasma and PBMCs are collected, washed with PBS and stored in freezing medium (10% DMSO and 90% Fetal Calf Serum). PBMCs are isolated with Sepmate or lymphoprep tubes. Cells are resuspended at 0.25 to 1×10⁷/mL and aliquoted in 500-1000 μL volumes in cryovials. Cryovials are stored in a Mr. Frosty container for 24 hours at −80 °C and then moved to liquid nitrogen freezer.

Serum and ctDNA—Blood samples are collected a 7-10 cc red top tube for serum and a 7-10 cc Streck tube for ctDNA. Blood in the serum collection tube should be allowed to clot for 30-60 minutes and then centrifuged to separate the serum. Serum should be stored in 2 mL cryovial tubes (Nalgene Cat #5000-0020) in 250 to 500 μL aliquots. Samples should be labeled with Biospecimen code number and the date and time of draw. Serum and ctDNA samples should be stored at −80 °C.

Sample labeling—All bone marrow and blood samples and clinical information before banking are coded and labeled with a unique PIN code (Patient Identification Number), TMI, a four-digit number followed by type of sample (PBMC: PBMC, P: Plasma, S: Serum) followed by sequence code (1: Prior to ATG, 2: Pre-TMI 2, 3; and 3: Post-TMI+cyclophosphamide). For example: TMI-0001-PBMC1 would be the first enrolled patient's PBMC sample take prior to ATG. Specimens will also be dated.

Analyses-Individual assays performed at core facilities depend on instrument availability and comparative cost of assays. If improved techniques and/or bioinformatic analyses become available to assess genomic, epigenetic, transcriptional, histological or other biological differences, we will incorporate these into our studies. Biospecimens and sample preparation follow the following procedure:

TCR sequencing: PBMCs are isolated and RNA extracted using Trizol Reagent. TCR cDNA libraries are generated by 5′RACE and sequenced on an Illumina platform.

T cell immunophenotyping: PMBCs are immunophenotyped using a T cell functional panel (TIGIT, CD3, FOXP3, TIM3, PD-1, OX40, CTLA4, LAG3, CD56, ICOS, Ki67, CD8, CD25, CD45 and CD4) as well as a Myeloid panel (CD141, CD14, CD11c, CD274, CD1c, CD206, HLA-DR, MerTK, CD86, CD11b, CD45 and CD15).

Serum cytokines: Serum or plasma cytokines may be assessed using Codeplex panels on the Isolyte system. These panels target up to 30 cytokines assessing inflammatory chemokines, cytokines and growth factors per sample.

Immune cell functionality: T cell function is assessed using single cell secretome chips for Human Adaptive Immune Panels using the Isolyte System from Isoplexis. In addition, macrophage and NK cell function is assessed using Human Innate Immune Panels.

Further circulating immune functions may be assessed using single cell RNAseq of PBMCs to assess changes in the subpopulations of circulating immune cells. Quantitative differences in immune cell subsets is assessed using fluorescent cytometry.

Additional correlative analyses may be conducted to understand features of donor leukocytes and anti-viral Tcm. Further biomarker assessments, such as gene expression profiling, clonal hematopoiesis, whole exome sequencing, or single-cell mass cytometry (CyTOF), may be conducted on peripheral blood tumor cells to correlate disease features and transplant efficacy.

Statistical Considerations, Design Operating Characteristics and Implementation—done as described in Example 1 of International Patent Application Publication No. WO2021/024264.

Secondary endpoints, data analyses, reporting requirements and adverse events—done as described in Example 1 of International Patent Application Publication No. WO2021/024264.

Background Drug Information—as described in Example 1 of International Patent Application Publication No. WO2021/024264.

### Example 2

### Production of Anti-Viral Central Memory CD8⁺ Veto Cells

### Manufacturing and Characterization Information of the Cell Preparations

### Cell Type and Derivation

This protocol requires the collection of two separate stem cells preparations. One type of stem cell preparation is from a mobilized haploidentical donor, part of which is selected for CD34⁺ cells, and the other part depleted of CD3⁺/CD19⁺ cells using the Miltenyi CliniMACS® device. The second type of preparation is an allogeneic non-mobilized peripheral blood mononuclear (PBMC) cells for the production of the anti-viral central memory CD8⁺ veto T cells. The PBMC are used to generate dendritic cells and memory responder T cells (CD4⁻CD56⁻CD45RA⁻), which are then co-cultured to stimulate production of anti-viral central memory CD8⁺ veto cells (Tcm). The Tcm cells are then expanded, harvested and infused. This preparation is used in the manufacture and infusion of Tcm cells, to achieve engraftment without GVHD after the infusion of T cell depleted megadose haploidentical donor from CD34⁺ enriched cells.

### Manufacturing Laboratory

Cell preparations of some embodiments of the invention are processed in the Department of Stem Cell Transplantation and Cellular Therapy (SCTCT) Cell Therapy Laboratory (CTL) at MDACC. This facility contains both a Cell Processing Laboratory for minimally manipulated cell preparations and Classified Suites (Class ISO 7) for more than minimally manipulated cell preparations. The cell preparation of some embodiments of the invention is manufactured primarily in the Class ISO 7 suites. The CTL also includes a Flow Cytometry and Quality Control Laboratories for the support of clinical trials, research, and development.

### Relevant Accreditations (FACT, CAP, CLIA)

The SCTCT CTL is registered with the FDA (FEI #0001670014) and is accredited by the Foundation of Accreditation of Cellular Therapy (FACT), the College of American Pathologists (CAP), and holds a Clinical Laboratory Improvement Amendments (CLIA) Certificate for Accreditation issued by the Centers for Medicare and Medicaid Services (CMS).

### Starting Material

### PBMC:

Two separate cell type preparations are required for this protocol. One preparation comprises an allogeneic non-mobilized peripheral blood mononuclear cells for the production of the anti-viral central memory CD8⁺ veto T cells (Tcm) while the other is from a mobilized haploidentical peripheral blood preparation. Both preparations are collected via leukapheresis.

The non-mobilized (unprimed) peripheral blood mononuclear cells for production of the Tcm cells are collected about 8 days before the planned transplant date (Day 0). The target leukapheresis mononuclear cells yield is of 1×10¹⁰. The Tem cells are infused on D+7 after infusion of the CD34⁺ Enriched Preparation.

The mobilized peripheral blood cells are collected over 2 days: D-2 and D-1. The cells collected on D-2 undergo CD34⁺ selection and infusion to the patient on D-1. The cells collected on D-1 are depleted of CD3⁺/CD19⁺ cells and infused to the patient on D-0. Multiple collections can be pooled prior to the enrichment procedure. A back up fraction of unmodified PBMC containing

2×10⁶ CD34⁺ cells per kg ideal body weight is taken from the day 1 collection and cryopreserved.

### Process Description

MDACC donors are first assessed for suitability including eligibility screening and physical examination. Donors are then consented and scheduled for collection. Target cell numbers are protocol specific and multiple collections may be required. After the collection procedure is complete, cells are transported from the harvesting facility to the CTL Laboratory by CTL staff in plastic coolers to protect the cells from temperature fluctuations and physical damage. Preparations are then logged at the CTL.

Infectious Disease Testing and Prevention of Cross-Contamination (including Donor Eligibility, Medical History, List of Testing, Donor Eligibility Undetermined, Release of Cell Preparations from Ineligible Donor, Infusion of Cell Preparations with Positive IDM Results)—as described in Example 2 of International Patent Application Publication No. WO2021/024264.

### Cell Processing

### Generation of the Anti-Viral Central Memory CD8⁺ Veto Cells (Tcm Cells)

### Procedure Overview

The collection of unprimed peripheral blood mononuclear cells for production of the anti-viral central memory CD8⁺ veto T cells are collected 8 days before the planned infusion date (Day 0). The veto cells are infused on D+7 post megadose T Cell Depleted Cells.

As illustrated in FIG. 2:

### Days −8 to −7: Collection of Unprimed Leukapheresis, MNC Isolation and CD14⁺ Cells Selection

### PBMC Isolation, Thrombowash:

The non-mobilized leukapheresis cell preparation of one embodiment is diluted at 1:2 with Dulbecco's Phosphate-Buffered Saline (DPBS) without Calcium and Magnesium supplemented with 0.5% of Human Serum Albumin (HSA). Samples for cell count, Trypan Blue (TB) viability and sterility are obtained prior to ficoll process. The MNC are isolated by ficoll density gradient separation. After ficoll, the MNC cell preparation of one embodiment is depleted of platelet (thrombowash) twice by manual centrifugation prior to CD14⁺ isolation and resuspended with Wash Buffer. Samples for cell counts and TB viability are removed for QC testing.

Following the Post-Ficoll Thrombowash, the cells are divided into two equal fractions and diluted up to approximately 500 mL each. One half are processed for the Dendritic Cell (DC) isolation by CD14⁺ Selection and the other half (Fraction I) are kept overnight for the CD8⁺ Memory T Cell Enrichment process the following day.

Fraction I is centrifuged and resuspended at a concentration of 30×10⁶ cells/ml in T Cell Growth Media (Click's Media with advanced RPMI 1640 supplemented with 1:100 Glutamaxe and 5% Human AB Serum) along with IL-7 (30 IU/mL). Samples for sterility testing are removed. Fraction I is then plated onto tissue culture flasks and incubated overnight at 37 °C, 5% CO₂.

### DC Isolation—CD14⁺ Monocyte Isolation and Adherence

The DC isolation fraction is centrifuged and resuspended in 50 ml of Magnetic Bead Buffer (Dulbecco's Phosphate-Buffered Saline (DPBS) without Calcium and Magnesium supplemented with 0.6% ACD-A and 0.5% of HAS). Samples for cell count and TB viability are removed.

The DC Isolation MNC fraction is incubated with the CD14 Reagent (CD14 monoclonal antibodies conjugated to super-paramagnetic iron dextran particles). The cells are then washed with Magnetic Beads buffer to remove excess reagent. Samples for cell count, TB viability and immunophenotyping are removed. Following the wash, CD14 labeled cells are processed on the SuperMACS™ II using the XS Separation Column per established SOP. The magnetically labeled cells (CD14⁺) are retained by the column and the CD14 negative cells are removed. The CD14⁺ cells are then released from the column and collected. The CD14⁺ fraction is washed and resuspended in DCs medium (CellGro/1% HSA), while CD14⁻ fraction is washed and resuspended in T cell growth medium. Samples from each fraction are removed for cell count, TB viability and immunophenotyping.

The CD14⁺ enriched cell preparation of one embodiment is resuspended at a cell concentration of 3×10⁶ cells/ml in DC Medium (CellGro/1% HSA) supplemented with IL-4 (1000 IU/mL) and GM-CSF (2000 IU/mL). Samples for sterility testing are removed. The cell suspension is then seeded in Cell Factory plates and incubated overnight for 16-24 hours in at 37 °C, 5% CO₂.

The CD14 negative cell concentration is adjusted at 30×10⁶ cells/ml in T Cell Growth Media along with IL-7 (30 IU/mL). Samples for sterility testing are removed. The CD14 Negative Fraction is then plated onto tissue culture flasks and incubated overnight at 37 °C, 5% CO₂. These cells are combined on the next day with Fraction I for the CD8⁺ Memory T Cell Enrichment process.

### Day −6: Isolation of Memory Cell and DC Maturation Induction

### DC Maturation Induction

After 24 hours of incubation of the CD14⁺ Enriched cells, cytokines: IL-4 (1000 IU/mL), GM-CSF (2000 IU/mL), LPS (40 ng/mL), and IFN-γ (200 IU/mL) are added to the Cell Factories to induce maturation of the DCs. The cells are then incubated with the cytokines at 37 °C, 5% CO₂ for 16 hours (+/−2 hours).

### Isolation of Memory Cell

After the overnight storage, the cells in Fraction I and in the CD14 Negative fraction are harvested and combined. Once combined (T Cell Isolation TNC Fraction), the cells are centrifuged and resuspended in CliniMACS®/0.5% HSA Buffer to a minimum of 1:2 ratio. Platelet depletion (thrombowash) is performed by centrifugation and resuspension of the cells pellet in CliniMACS®/0.5% HSA. A sample for cell count and TB viability is removed.

The post-platelet depleted cell preparation of one embodiment is incubated with IVIg for 10-15 minutes. After the initial incubation, the anti-CD4, anti-CD56 and anti-CD45RA reagents (anti-CD4, anti-CD56 and anti-CD45RA antibodies conjugated to super-paramagnetic particles) are added to the cell preparation and incubated for 30 minutes on an orbital rotator. At the end of the incubation the cells are washed by centrifugation and the cell pellet resuspended in CliniMACS®/0.5% HSA buffer to remove excess reagent. Cell count and immunophenotyping samples are removed. The CD4⁺/CD56⁺/CD45RA⁺ labeled cells are then processed on the CliniMACS® using the depletion tubing set and the depletion program. In this case, the magnetically labeled cells (CD4⁺, CD56⁺ and CD45RA⁺) are retained by the column and CD4, CD56 and CD45RA negative cells pass through the column and are collected as a CD4⁻CD56⁻CD45RA-Depleted Fraction (Fraction II). Fraction II is washed and resuspended in T cell Growth Medium. Samples from each fraction are removed for cell count, TB viability and immunophenotyping. Once the volume and TNC of each fraction is determined, the positive fraction is discarded and the negative fraction (Fraction II) is further processed.

Fraction II cell concentration is adjusted at 2×10⁶ cells/ml in T Cell Growth Media along with IL-7 (30 IU/mL). Samples for sterility testing are removed. Fraction II is seeded in G-Rex®100 and incubated at 37 °C, 5% CO₂ for 24 hours (+/−2 hours).

### Limitations:

The maximum TNC to process with one reagent kit and tubing set is 200×10⁶ TNC/mL unless higher number is approved by the Laboratory Director or designee. The maximum load volume for the CliniMACS® instrument is 300 ml.

### Day −5: Mature DC Harvest, Viral Peptide Loading and Co-Culture

Mature DC (mDC) Harvest

After the 16 hour incubation, the supernatant is removed and the Cell Factories are gently washed with warm Magnetic Bead Buffer. The washing buffer and any non-adherent cells are removed and added to the mDC supernatant. Samples from the mDC supernatant and wash buffer are removed for cell count, TB viability and immunophenotyping.

The adherent mDC are detached and harvested from the Cell Factories by adding ice-cold Magnetic Bead buffer and left resting for 30 minutes on frozen gel packs. After the 30 minutes, the mDC are harvested and the Cell Factories are washed once with ice-cold Magnet bead buffer. The Cell Factories are inspected microscopically to determine whether all the mDCs are removed. If it is observed that not all the mDCs are removed the wash process is repeated. The harvested (adherent) mDCs suspension is then centrifuged, washed and resuspended in Magnetic Bead Buffer. Samples from the adherent mDCs are removed for cell count, TB. After the mDCs volume and TNC is determined the cell concentration is adjusted to 1×10⁷ cells/ml for peptide loading. In-process samples for immunophenotyping are removed. Once the volume and TNC of each fraction is determined the mDC supernatant fraction is discarded and the mDCs cell preparation of one embodiment is further processed.

### Loading of Viral Peptides

After mDCs are harvested, the calculated peptivators (AdV5 Hexon, HCMV pp65, EBV select and BKV LT) are added to the cells and are incubated for 1 hour at 37 °C, 5% CO₂. Following the incubation the viral peptide loaded mDCs are washed and centrifuged with Magnetic Bead Buffer. Then the viral peptide loaded mDCs are resuspended with T cell Growth Medium and irradiated with 30-25 Gy via X-Ray source.

Once irradiation is complete the viral peptide loaded mDCs are washed, centrifuged and resuspended in T Cell Growth Medium. Samples are removed for cell count, TB viability, immunophenotyping and sterility.

### Days −5 to −2: Co-Culture Preparation and Starvation Phase

Following the 24 hour incubation of Fraction II (CD4⁻CD56⁻CD45RA⁻), the cells are retrieved from the incubator, centrifuged and resuspended in T Cell Growth Medium. Samples for cell count, TB viability, immunophenotyping and sterility are removed. The cells from Fraction II (CD4⁻CD56⁻CD45RA⁻), are then washed and seeded in G-Rex®100 at a concentration of 1×10⁶ cells/ml (100 ml/G-Rex®100) together with the viral peptide loaded mDCs at a ratio of 5:1 (Fraction II Cells: Dendritic Cells (DC)) in T Cell Growth Media along with IL-21 (100 IU/mL). The co-culture cells are incubated for 3 days in 37 °C, 5% CO₂ for the starvation period.

### Days −2 to +6: Differentiation and Expansion of Anti-Viral Veto Cells (Tcm)

Following the 72 hour starvation phase, a sample from the supernatant of the G-Rex®100 is carefully removed without disrupting the cell layer, to determine the pH and glucose level of the culture. The pH should be at the physiologic range (pH 7.2-7.6) and the glucose at least 50 mg/dl. Fresh T cell growth medium supplemented with IL-7 (30 IU/mL), IL-15 (125 IU/mL) and IL-21 (100 IU/mL) is added to each G-Rex®100 at 50% of the culture volume. The cells are then incubated for an additional 48 hours at 37 °C with 5% CO₂. This process is repeated every 48 hours until the end of culture on day +7.

The cells are in culture for up to 12 days. Fresh media containing cytokines or only cytokines are added every 48 hours depending on the pH and glucose level. If the glucose level is between:
Glucose Level of 170 mg/dL to 130 mg/dL: only cytokines IL-7, IL-15, IL-21 are added to the culture.
Glucose Level of 129 mg/dL to 100 mg/dL: 100 ml (25% of the G-Rex®100 volume) of fresh T cell growth medium+cytokines IL-7, IL-15, IL-21 are added to the culture.
Glucose Level of 99 mg/dL to 50 mg/dL: 200 ml (50% of the G-Rex®100 volume) of fresh T cell Growth medium+cytokines IL-7, IL-15, IL-21 are added to the culture.

Of note: The maximum volume level in the G-Rex®100 is 400 mL. The volume of culture is replenished if the maximum volume in the G-Rex®100 is reached.

### Day +4:

On day +4, a sterility sample is obtained for quality control release testing.

### Day +6: Culture Cell Count Assessment

On Day +6 a cell count, TB viability and immunophenotyping sample is removed for TNC determination.

### Day +7: Final Formulation and Infusion of Anti-viral CD8 Central Memory Cells (Tcm)

On Day +7, the Tem cells, inspected for visual contamination, harvested and washed with cell suspension media (Plasmalyte-A/0.5% of HSA). Samples are removed for non-release testing: cell count, TB viability, sterility and release-testing: endotoxin and mycoplasma prior to wash of the cells.

After the cell preparation of one embodiment is washed and resuspended, release testing samples for cell count, viability, and immunophenotyping are removed. Following the cell count and TNC determination, the cell preparation of one embodiment is resuspended in approximately 50-100 mL of cell suspension media to a Tcm cell dose according to the dose-prescribed in the clinical protocol. Samples for release testing are obtained for gram stain and sterility as non-release testing. The final Tcm cell preparation meets release criteria prior to release of the cell preparation.

The final Tcm cells are transported from the CTL to the patient floor by the CTL staff in a plastic cooler to protect the cell preparation from temperature fluctuations and physical damage. The CTL Staff deliver and issue the cell preparation to infusing personnel.

### Generation of the CD34⁺ Enriched Cells

### Procedure Overview

The mobilized hematopoietic progenitor cells (also termed HPC-A) is collected in two days (D-2 and D-1). A back-up fraction of unmodified PBMC containing 2×10⁶ CD34⁺ cells per kg ideal body weight is set aside and cryopreserved. The remaining D-2 collection is kept overnight, CD34⁺ selected and infused on D-1. The second day's collection (D-2) is kept overnight, depleted of CD3⁺/CD19⁺ cells and infused on D0. The maximum T cell dose in the entire T cell depleted stem cell transplant collected from all fractions is 2×10⁵ CD3⁺ cells per kg ideal body weight.

Both procedures are performed in the CTL Core Laboratory using the Miltenyi CliniMACS® device. The CTL has well established SOPs for the use of this device and has used it in the manufacture of cell preparations for multiple clinical trials. Single HPC-A preparations may be split or pooled depending on cell number and scheduling.

### CD34⁺ Enrichment Process

### Setup

CliniMACS® PBS/EDTA Buffer bags (1000 ml) containing 0.5% Human Serum Albumin (HSA) are prepared prior to processing according to well-established laboratory SOP. The weight of the HPC-A preparation is determined. Samples from Day-collection are removed for the following tests: cell count, viability, sterility, immunophenotyping. Each buffer and cell preparation bag is labeled with Patient's name, MDACC number, and date of preparation.

### CD34⁺ Isolation

The cells collected in D-2 of one embodiment are incubated with the CD34 reagent (CD34 antibody conjugated to super-paramagnetic particles). The cells are washed to remove excess reagent. Cell count and immunophenotyping samples are removed. The CD34 labeled cells are then processed on the CliniMACS® using the CliniMACS® tubing set and the CD34⁺ selection program. In this case, the magnetically labeled cells (CD34⁺) are retained by the column and CD34 negative cells are removed. The CD34⁺ cells are then released from the column and collected. Samples from each fraction are removed for cell count, viability and immunophenotyping.

The CD34⁺ enriched cell preparation may be cryopreserved for infusion (as discussed below) or may be used as fresh cells.

### Limitations:

The maximum TNC to process with one reagent kit and tubing set is 6×10¹⁰ unless higher number is approved by the Laboratory Director or designee. The maximum load volume for the CliniMACS® instrument is 300 ml.

CD3⁺/CD19⁺ Depletion Process

### Setup

CliniMACS® PBS/EDTA Buffer bags (1000 ml) containing 0.5% Human Serum Albumin (HSA) is prepared prior to processing according to well-established laboratory SOP. The weight of the HPC-A cell preparation is determined. Samples from the HPC-A are removed for the following tests: cell count, viability, sterility, and immunophenotyping. Each buffer and cell preparation bag is labeled with Patient's name, Medical Record Number (MRN), and date of preparation.

### Platelet Depletion

The HPC-A cell preparation of one embodiment is platelet depleted prior to the CD3⁺/CD19⁺ depletion using the COBE 2991. Once the cells are removed from the COBE 2991, samples for cell count are removed. The cell preparation is then incubated with the CD3 reagent and CD19 reagent.

CD3⁺/CD19⁺ Depletion

The post-platelet depletion HPC-A cell preparation of one embodiment is incubated with IVIg for 10-15 minutes. After the initial incubation the CD3⁺ and CD19⁺ reagents (CD3 and CD19 antibodies conjugated to super-paramagnetic particles) are added to the cell preparation and incubated for 30 minutes on an orbital rotator. At the end of the incubation, the cell are washed using the COBE 2991 to remove excess reagent. Cell count and immunophenotyping samples are removed. The CD3⁺/CD19⁺ labeled cells are then processed on the CliniMACS® using the depletion tubing set and the depletion program. In this case, the magnetically labeled cells (CD3⁺/CD19⁺) are retained by the column and CD3 and CD19 negative cells pass through the column and are collected as a CD3⁺/CD19⁺ depleted fraction. Samples from each fraction are removed for cell count, viability and immunophenotyping.

The CD3⁺/CD19⁺ depleted cell preparation of one embodiment is cryopreserved for infusion (as discussed below) or may be used as fresh cells.

Limitations: The maximum TNC to process with one reagent kit and tubing set is 8×10¹⁰ unless higher number is approved by the Laboratory Director or designee.

2^{nd} CD3⁺ Depletion (not a required step, to be used as needed)

If more than 2×10⁵ CD3/kg ideal body weight are present, a second cycle of CD3 depletion may be performed on the CD3⁺/CD19⁺ depleted to further reduce the CD3 population in the cell preparation. Samples for cell counts and immunophenotyping, viability and sterility are obtained from each fraction.

Alternatively infusion of only part of the T cell depleted fraction is utilized so as to avoid infusion of more than 2×10⁵ CD3 cells/kg ideal body weight.

Optional Cryopreservation of CD34⁺ Enriched and CD3⁺/CD19⁺ Depleted Cell Preparations:

After cell selection or depletion and sampling, the cell preparations of one embodiment are concentrated and cryopreserved according to procedures validated at the CTL. A final cell count, viability, immunophenotyping, gram stain and sterility sample is obtained prior to cryopreservation of the cell preparation. Following the cell count and TNC determination, the cell preparation of one embodiment is cryopreserved in freeze media (50% Plasma-Lyte A, 7.5% DMSO, and 35% HSA 25%) according to SOP. Bags containing the CD34⁺ Enriched cells or CD3⁺/CD19⁺ depleted cells are frozen in a controlled rate freezer and stored in a liquid nitrogen freezer in vapor phase.

Of note: The final cell preparation is the preparation before cryopreservation as the thawing of the cells occurs at bedside without further manipulation.

### Cell Preparation Release and Infusion

On the day of infusion, the CD34⁺ Enriched cells and/or CD3⁺/CD19⁺ Depleted cell preparations are delivered to the patient floor by the CTL staff and issued to the infusing personnel. The final cell preparations meet lot release criteria prior to release of the cell preparations. These cells are then infused into the patient according to protocol specific dose. Of note, when cryopreserved are used, the cells are thawed on the floor and released to infusing personnel per standard operating procedure. Also, when cryopreserved are used, the final cell preparations are transported from the CTL to the floor by CTL staff in an approved styrofoam LN2 transportation container with seamless metal inserts that are be filled with approximately 2 inches of liquid nitrogen. All cell preparation testing is protocol specific and preformed according to SOPs.

Reagents—as described in Example 2 of International Patent Application Publication No. WO2021/024264.

Testing of cell preparations—as described in Example 2 of International Patent Application Publication No. WO2021/024264.

### Cell Preparation Release Criteria Testing and Additional Testing

TABLE 3
Tcm Preparation Lot Release testing and Specifications

| Test | Specifications |
|---|---|
| Viability (7AAD) | ≥70% |
| Visual Inspection | No evidence of contamination |
| Gram Stain | No organism seen |
| Sterility (Day 12) | Negative-to-Date |
| Mycoplasma | Negative |
| (MycoAlert) | |
| Endotoxin (LAL) | <5 EU/Kg ideal body weight |
| Immunophenotyping | ≥30% CD3⁺CD8⁺CD62L⁺CD45RO⁺7AAD⁻ of |
| | CD45⁺ cells |
| CD8⁺ Cell Dose | Dose 1: 2.5 × 10⁶ CD8⁺/kg ideal body weight |
| | Dose 2: ≤5 × 10⁶ CD8⁺/kg ideal body weight |
| | but ≥2.6 × 10⁶ CD8⁺/kg ideal body weight |
| | Dose 3: ≤10 × 10⁶ CD8⁺/kg ideal body weight |
| | but ≥5.1 × 10⁶ CD8⁺/kg ideal body weight |

TABLE 4
CD34⁺ Enriched Preparation Lot Release testing and Specifications

| Test: | Specifications: |
|---|---|
| Sterility | No Growth at 14 Days |
| Gram Stain | No Organisms Seen |
| Viability (Final Preparation) | ≥70% |
| Total CD3⁺ Cell Dose | <2 × 10⁵ CD3⁺ cells per kg ideal |
| | body weight |
| Total CD34⁺ Cell Dose | ≥5 × 10⁶ CD34⁺ cells per kg ideal |
| | body weight |

TABLE 5
Additional In-Process Test (non-release)

| Test: | Specification |
|---|---|
| Sterility | No Growth at 14 Days |
| Immunophenotype | For Information Only |
| TNC | For Information Only |
| Mycoplasma (PCR) | Negative |

### Example 3

Total Marrow Irradiation Combined with Anti-Viral Central Memory CD8+ Veto Cells in Haploidentical Stem Cell Transplantation for Treating AML and MDS Cancers

For production of the veto cells, donor memory CD45RO+ CD8 T cells were isolated and co-cultured with donor dendritic cells pulsed with peptides from four viruses (EBV, CMV, BKV, and Adenovirus). The culture was carried out under cytokine deprivation for 3 days to allow death by neglect of anti-host clones, then IL15, IL21 and IL7 were added to allow expansion of anti-viral central memory CD8 T cells. In 11 runs, >1×10¹⁰ CD45RO+CD3+CD8+CD62L+ T cells were generated from a single leukapheresis. At the end of 12 days of culture, there were 96.4±2.5% CD3+CD8+ T cells of which 80.1±10.6% (range 71.5 to 92.8) exhibiting a CD62L+CD45RO+ CM phenotype. Limiting dilution analysis revealed >3 log depletion of alloreactive T cell clones. Anti-viral activity tested by intracellular expression of TNF-α and INF-g showed an average of 38.8±19.6% positive cells upon 6 hours stimulation against the viral peptide mixture.

Three patients (two with AML and one with MDS, see Table 6 hereinbelow) received anti-thymocyte globulin 2 mg/kg daily on Day −8 to −6, Fludrabine 30 mg/m2 on Day −5 to −2 and 12 Gy total marrow irradiation combined with spleen irradiation, over 4 consecutive days (as described in details in Example 1 hereinabove and shown in FIG. 1). Two collections of donor G-CSF mobilized peripheral blood progenitor cells were performed on day 0 and day 1. The first infusion was CD34 selected cells and the second was depleted of CD3+/CD19+ cells. The infusions included 1 to 2×10 CD3+ T cells/kg. Cyclophosphamide (CY) 50 mg/kg was given on day 3 and 4. Veto cells (5×10/kg) were infused on Day 7.

All three patients engrafted (see Table 6 hereinbelow). One of the AML patients developed steroid-responsive rash which might represent overall grade 2 GVHD of the skin although skin biopsy suggest allergic response to a drug. No GVHD could be detected in the second AML patient or the MDS patient. All patients had a detectable and significant donor myeloid and T cell chimerism at all time points tested (see Table 7 hereinbelow).

In conclusion, the data demonstrate reliable engraftment of haploidentical TCD HSCT combined with anti-viral CM veto CD8 T cells following a well-tolerated reduced intensity conditioning comprising TMI and show low rates of GVHD in the absence of immunosuppression beyond CY treatment on day 4.

TABLE 6
Engraftment (>0.5 ANC), GVHD, Discharge and relapse dates
following transplantation of T cell depleted megadose HSCT
with 5 million/kg anti-viral central memory veto cells

| January 2023 | Pt #7 | Pt #9 | Pt #10 |
|---|---|---|---|
| Disease | AML | MDS | AML |
| Age | 70 | 73 | 73 |
| GVHD | Grade 2 skin rush | 0 | 0 |
| | responsive to steroids. | | |
| | Biopsy suggestive of | | |
| | potential allergic | | |
| | response to a drug | | |
| Day of relapse | — | — | — |
| Day of death/alive | Alive and well (D276) | Alive and | Alive |
| | | well (D115) | (D125)* |
| Day of engraftment | 16 | 17 | 18 |
| Day of discharge | 21 | 30 | 30 |

*Recovered from autoimmune neutropenia and awaiting recovery from thrombocytopenia

TABLE 7
Myeloid an T cell chimerism

| | January 2023 | | | | | |
|---|---|---|---|---|---|---|
| | Pt #7 | | Pt #9 | | Pt #10 | |
| | Disease | | | | | |
| | AML | | MDS | | AML | |
| | % | Day | % | Day | % | Day |
| Myeloid | 100 | 32 | 100 | 29 | 100 | 60 |
| Chimerism | 100 | 60 | 100 | 60 | 100 | 102 |
| | 100 | 118 | | | | |
| T cell | 61 | 30 | 100 | 29 | 32 | 60 |
| Chimerism | 97 | 60 | 100 | 60 | 25 | 102 |
| | 92 | 118 | | | | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of treating a disease in a subject in need thereof, wherein said disease comprises pathological cells residing in a bone marrow of said subject, the method comprising:
(a) conditioning the subject under a pre-transplant conditioning protocol comprising a therapeutically effective amount of total marrow irradiation (TMI); and subsequently
(b) transplanting into said subject therapeutically effective amount of T cell depleted immature hematopoietic cells; and subsequently
(c) administering to said subject a therapeutically effective amount of cyclophosphamide; and subsequently
(d) administering to said subject a therapeutically effective amount of veto non-graft versus host disease (GVHD) inducing cells,
thereby treating the disease in the subject.

2. (canceled)

3. The method of claim 1, wherein said disease is cancer, optionally wherein said cancer comprises a myeloid malignancy or multiple myeloma, optionally wherein said myeloid malignancy is selected from the group consisting of AML, CML and MDS, optionally wherein said cancer comprises AML or MDS.

4-6. (canceled)

7. The method of claim 1, wherein said disease is non-cancerous, optionally wherein said disease is selected from the group consisting of sickle cell anemia, aplastic anemia, thalassemia and metabolic genetic disease.

8. (canceled)

9. The method of claim 1, wherein said veto non-GVHD inducing cells are obtained from the same donor as said T cell depleted immature hematopoietic cells.

10. The method of claim 1, wherein said conditioning further comprises a therapeutically effective amount of spleen irradiation.

11. The method of claim 1, wherein said TMI is administered on days −7 to −1 prior to transplantation of said T cell depleted immature hematopoietic cells;
wherein said therapeutically effective amount of said TMI comprises a total of 8-18 Gy;
wherein said therapeutically effective amount of said TMI is administered in at least 2 doses administered on consecutive days; and/or
wherein said therapeutically effective amount of said TMI is administered in 4 doses administered on consecutive days.

12-14. (canceled)

15. The method of claim 1, wherein said T cell depleted immature hematopoietic cells are derived from a donor non-syngeneic to said subject.

16. The method or the combination for use of claim 15, wherein said non-syngeneic is allogeneic, optionally wherein said allogeneic donor is an HLA matched sibling, an HLA matched unrelated donor, an HLA haploidentical related donor or a donor displaying one or more disparate HLA determinants.

17. (canceled)

18. The method of claim 1, wherein said therapeutically effective amount of said T cell depleted immature hematopoietic cells comprises less than 5×10⁵ CD3⁺ T cells per kilogram ideal body weight of said subject, optionally wherein said therapeutically effective amount of said T cell depleted immature hematopoietic cells comprises less than 2×10⁵ CD3⁺ T cells per kilogram ideal body weight of said subject;
wherein said therapeutically effective amount of said T cell depleted immature hematopoietic cells comprises at least 5×10⁶ CD34⁺ cells per kilogram ideal body weight of said subject; and/or
wherein said therapeutically effective amount of said T cell depleted immature hematopoietic cells are depleted of CD3⁺ and/or CD19⁺ expressing cells.

19-21. (canceled)

22. The method of claim 1, wherein said therapeutically effective amount of said cyclophosphamide comprises 25-200 mg cyclophosphamide per kilogram ideal body weight of said subject;
wherein said therapeutically effective amount of said cyclophosphamide is administered to said subject in two doses between days 2 and 5 following said transplantation of said T cell depleted immature hematopoietic cells;
and/or
wherein said therapeutically effective amount of said cyclophosphamide is administered to said subject in two doses 3 and 4 days following said transplantation of said T cell depleted immature hematopoietic cells.

23-24. (canceled)

25. The method of claim 1, wherein said veto non-GVHD inducing cells comprise a central memory T-lymphocyte (Tcm) phenotype.

26. The method of claim 1, wherein said veto non-GVHD inducing cells have an anti-viral activity.

27. The method of claim 1, wherein said veto non-GVHD inducing cells are obtainable by:
(i) contacting a first population of peripheral blood mononuclear cells (PBMCs) from a donor with an antibody capable of binding CD14⁺ expressing cells and selecting CD14⁺ expressing cells capable of maturing into antigen presenting cells;
(ii) loading said antigen presenting cells with a viral peptide;
(iii) treating a second population of PBMCs of the same donor as said first population of PBMCs with one or more agents capable of depleting CD4⁺, CD56⁺ and CD45RA⁺ expressing cells so as to obtain a population of cells comprising T cells enriched in memory T cells expressing a CD45RA⁻CD8⁺ phenotype;
(iv) contacting said population of cells comprising said T cells enriched in said memory T cells with said antigen presenting cells loaded with said viral peptides of step (ii) in the presence of IL-21 so as to allow enrichment of viral reactive memory T cells; and
(v) culturing said cells resulting from step (iv) in the presence of IL-21, IL-15 and/or IL-7 so as to allow proliferation of cells comprising said Tem phenotype.

28. The method of claim 1, wherein said therapeutically effective amount of said veto non-GVHD inducing cells is administered on day 6-9 following said transplantation of said T cell depleted immature hematopoietic cells.

29. The method of claim 1, wherein said therapeutically effective amount of said veto non-GVHD inducing cells comprises at least 2.5×10⁶ CD8⁺ cells per kg ideal body weight of said subject.

30. The method of claim 1, wherein said subject is not treated chronically with GVHD prophylaxis following said transplantation.

31. The method of claim 1, wherein said conditioning further comprises an anti-B cell therapy, optionally wherein said anti-B cell therapy comprises an anti-B cell antibody, optionally wherein said anti-B cell therapy comprises Rituximab.

32-33. (canceled)

34. The method of claim 1, wherein said conditioning further comprises T cell debulking, optionally wherein said T cell debulking is effected by antibodies, and optionally wherein said antibodies comprise at least one of an anti-thymocyte globulin (ATG) antibody, an anti-CD52 antibody and anti-CD3 antibody, optionally wherein said T cell debulking is effected by an anti-thymocyte globulin (ATG) antibody.

35-36. (canceled)

37. The method of claim 1, wherein said conditioning further comprises a chemotherapeutic agent, optionally wherein said chemotherapeutic agent comprises at least one of Fludarabine, Busulfan, Melphalan, Thiotepa and cyclophosphamide, optionally wherein said chemotherapeutic agent comprises Fludarabine.

38-39. (canceled)
